(19) United States
(12) Patent Application Publication
(10) Pub. No.: US 2022/0127565 A1
(43) Pub. Date: Apr. 28, 2022

(54) A Self-Assembling Short Amphiphilic Peptide And Related Methods And Uses

(71) Applicant: Agency for Science, Technology and Research, Singapore (SG)

(72) Inventors: Yihua Loo, Singapore (SG); Chwee Aun Andrew Wan, Singapore (SG); Chih Urn Benjamin Tai, Singapore (SG)

(21) Appl. No.: 17/429,132

(22) PCT Filed: Feb. 7, 2020
(86) PCT No.: PCT/SG2020/050060

§ 371 Date: Aug. 6, 2021

(30) Foreign Application Priority Data
Feb. 8, 2019 (SG) 10201901117W

(51) Int. Cl. C12N 5/0735; C07K 11/00

(52) U.S. Cl. CPC C12N 5/0606; C07K 11/00; C12N 2513/00; C12N 2539/00; C12N 2533/50

(57) ABSTRACT

There is provided a self-assembly amphiphilic peptide having the formula (I): XYZ (I), wherein X is a polar moiety at the N-terminus; X and Z each independently has between 1 to 4 residues of aliphatic amino acids or analogs or derivatives thereof, and wherein the average degree of hydrophobicity of the residues in block Z is more than the average degree of hydrophobicity of the residues in block Y. Disclosed are compositions and hydrogel comprising the peptide thereof. Also disclosed are methods of treatment for tissue regeneration, wound healing and methods of culture of stem cells, tissues and organoids.

### RELATED APPLICATIONS

This application is the U.S. National Stage of International Application No. PCT/SG2020/050060, filed Feb. 7, 2020, which designates the U.S., published in English, and claims priority under 35 U.S.C. § 119 or 365(c) to SG Application No. 10201901117W, filed Feb. 8, 2019. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file:

a) File name: 59751002001_SequenceListing_ST25.txt; created Dec. 20, 2021, 27,000 bytes in size.

### TECHNICAL FIELD

The present disclosure relates broadly to a peptide, such as an amphiphilic peptide, and related hydrogels, compositions, methods and uses.

### BACKGROUND

The self-assembly of well-defined biomacromolecules is emerging as a powerful strategy for large-scale biofabrication and tissue engineering. It offers reproducibility and versatility in the preparation of three-dimensional macromolecular structures with high levels of precision and complexity. Moreover, desirable attributes such as stimuli-responsiveness, adaptation, recognition and memory can be imbued through inclusion of specific domains.

In particular, short peptide motifs are excellent building blocks in view of their simple structure, facile synthesis and intrinsic biological properties (such as specificity, bioactivity and biodegradability). The resulting nanoscale assemblies (structures such as micelles, nanotubes, nanospheres, nanofibers and nanotapes) have been effectively harnessed for delivery of therapeutics, stimulation of tissue regeneration and development of in vitro models.

Peptide self-assembly is driven by secondary structure and intermolecular interactions, which are in turn dictated by peptide sequence. The conformation of the peptide backbone is an integral element of its secondary structure, while the side chains of the constituent residues stabilize the intra- and intermolecular interactions. Self-assembling alpha-helical peptide motifs are particularly sensitive to sequence mutations due to the inherent dynamic complexities of peptide folding and oligomerization in solution. Consequently, helical self-assembling motifs are mostly derived from natural protein structures such as coiled coils and collagen mimetics. Of these, the alpha-helical coiled coil is the most well-studied and defined by its distinctive heptad HPPHPPP repeat of hydrophobic (H) and polar (P) residues. Similarly, collagen-mimetics are homo- or heterotrimeric assemblies of (X-Y-glycine) strands.

Extensive computational and experimental studies have focused on shuffling and point substitutions (such as alanine scans) to identify the key positions that dictate intra- and inter-molecular interactions involved in stable macromolecular assembly and optimal packing. However, due to gaps in the understanding of the physicochemical determinants of peptide self-assembly, attempts to fit novel motifs (as structural templates) into existing computational simulations results in models that lack predictive value for generating de novo self-assembling helices. Moreover, the immense number of potential permutations constrains the feasibility of experimental verification to ascertain the optimum amino acid sequence for self-organization under physiological conditions. The complexity is further compounded by expanding the constituents to incorporate non-natural constituents such as D-amino acids, alpha-hydroxyacids, beta- and gamma-amino acids, as well as alkyl chains. Consequently, the identification of new self-assembling helical motifs is largely serendipitous.

Due to the reasons above, the development of new de novo self-assembling short peptides remains slow. In particular, in the biomedical domain, the available de novo self-assembling short peptides that possess desirable properties, and/or that are amenable to functionalization, remain limited.

Thus, there is a need to provide a peptide, such as an amphiphilic peptide, and related methods and uses that address or at least ameliorate one or more of the above problems.

### SUMMARY

In one aspect, there is provided an amphiphilic peptide having the formula (I): XYZ (I), wherein X is a polar moiety at the N-terminus; Y and Z each independently has between 1 to 4 residues of aliphatic amino acids or analogs or derivatives thereof, and wherein the average degree of hydrophobicity of the residues in block Z is more than the average degree of hydrophobicity of the residues in block Y.

In some examples, the polar moiety is selected from the group consisting of a polar functional group, a polar amino acid, and a small polar biomolecule.

In some examples, the polar functional group is selected from the group consisting of amine, acetyl, hydroxyl, thiol, maleimide, and acid; or the polar amino acid is selected from the group consisting of lysine, histidine, glycine, serine and aspartic acid; or the small polar biomolecule is selected from the group consisting of biotin, alcohol, and saccharide.

In some examples, the amphiphilic peptide comprises a depsipeptide analog.

In some examples, the depsipeptide analog comprises an α-hydroxy acid analog.

In some examples, the N-terminus is acetylated and/or C-terminus is amidated. In some examples, the aliphatic amino acids comprise D-amino acids.

In some examples, the chirality of each of the residues in Z is the same.

In some examples, Z comprises a residue of an aliphatic amino acid with hydrophobic side chain, or analogs or derivatives thereof.

In some examples, the amphiphilic peptide is no more than 7 residues in length.

In some examples, the amphiphilic peptide is capable of self-assembling into a hydrogel.

In some examples, the amphiphilic peptide is selected from the group consisting of:

KVI, KGAVLI, KGAVIL, KGAVIA, RVI, RGAVLI, RGAVIL, RGAVIA, HVI, HGAVLI, HGAVIL, HGAVIA, OrnVI, OrnGAVLI, OrnGAVIL, OrnGAVIA, DapVI, DapGAVLI, DapGAVIL, DapGAVIA, DabVI, DabGAVLI, DabGAVIL, DabGAVIA, KgAVLI, KGaVLI, KgAVIL, KGaVIL;

KGAVLI-NH₂, KGAVIL-NH₂, KgAVLI-NH₂, KGaVLI-NH₂, KgAVIL-NH₂, and KGaVIL-NH₂;

Ac-KVI-NH₂, Ac-KGAVLI-NH₂, Ac-KGAVIL-NH₂, Ac-KGAVIA-NH₂, Ac-RVI-NH₂, Ac-RGAVLI-NH₂, Ac-RGAVIL-NH₂, Ac-RGAVIA-NH₂, Ac-HVI-NH2, Ac-HGAVLI-NH₂, Ac-HGAVIL-NH₂, Ac-HGAVIA-NH₂, Ac-OrnVI-NH₂, Ac-OrnGAVLI-NH₂, Ac-OrnGAVIL-NH₂, Ac-OrnGAVIA-NH₂, Ac-DapVI-NH₂, Ac-DapGAVLI-NH₂, Ac-DapGAVIL-NH₂, Ac-DapGAVIA-NH₂, Ac-DabVI-NH₂, Ac-DabGAVLI-NH₂, Ac-DabGAVIL-NH₂, Ac-DabGAVIA-NH₂, Ac-KgAVLI-NH₂, Ac-KGaVLI-NH₂, Ac-KgAVIL-NH₂, Ac-KGaVIL-NH₂;

GAVLI, SGAVLI, SGAVIL, SGAVIA, TGAVLI, TGAVIL, TGAVIA, SGAVLI, SGAVIA, SgAVLI, SGaVLI, SgAVIA, and SGaVIA;

SGAVLI-NH₂, SGAVIA-NH₂, SgAVLI-NH₂, SGaVLI-NH₂, SgAVIA-NH₂, and SGaVIA-NH₂;

Ac-GAVLI-NH₂, Ac-SGAVLI-NH₂, Ac-SGAVIL-NH₂, Ac-SGAVIA-NH₂, Ac-TGAVLI-NH₂, Ac-TGAVIL-NH₂, Ac-TGAVIA-NH₂, Ac-SgAVLI-NH₂, Ac-SGaVLI-NH₂, Ac-SgAVIA-NH₂, Ac-SGaVIA-NH₂;

DVI, DGAVLI, DGAVIL, EVI, EGAVLI, EGAVIL, and EGAVIA; and

Ac-DVI-NH₂, Ac-DGAVLI-NH₂, Ac-DGAVIL-NH₂, Ac-EVI-NH₂, Ac-EGAVLI-NH₂, Ac-EGAVIL-NH₂, and Ac-EGAVIA-NH₂,

wherein Orn=ornithine, Dap=2,3-diaminopropionic acid, Dab=2,4-diaminobutyric acid, g=glycolic acid and a=L-lactic acid.

In another aspect, there is provided a composition or hydrogel comprising the amphiphilic peptide as described herein.

In some examples, the composition or the hydrogel as described herein, wherein amphiphilic peptide, the composition or the hydrogel has one or more properties selected from the group consisting of: stable, biocompatible, biodegradable, biomimetic, xenofree, injectable, thixotrophic, substantially non-mutagenic, substantially resistant to enzymatic degradation, responsive to stimulus, responsive to change in pH, responsive to change in salt concentration, responsive to change in temperature, compatible with bioprinting and has a storage modulus of at least 1 kPa.

In yet another aspect, there is provided the composition or the hydrogel as described herein for use in therapy.

In yet another aspect, there is provided a method of treating a subject in need of tissue regeneration, the method comprising administering the amphiphilic peptide, the composition or the hydrogel as described herein into the subject in need thereof.

In yet another aspect, there is provided a use of the amphiphilic peptide, the composition, or the hydrogel as described herein in the manufacture of a medicament for tissue regeneration.

In yet another aspect, there is provided a method of cell, tissue or organoid culture, the method comprising: culturing the cell, the tissue or the organoid in contact with the amphiphilic peptide, the composition, or the hydrogel as described herein.

In some examples, when single cells are seeded on or in the hydrogel, the hydrogel is more capable of promoting cell migration and/or generating single colony as compared to a hydrogel composed of peptides having sequences that are inverted from the sequences of said amphiphilic peptide.

In yet another aspect, there is provided the hydrogel as described herein when used in culturing stem cell, tissue or organoid.

### Definitions

The term “peptide” as used herein includes not only compounds that consist exclusively of amino acids attached to one another via peptide bonds, but also compounds that include one or more chemical modifications, for example to the amino acid residues themselves, the peptide bonds linking the residues together, and/or the termini of the peptide. The term “peptide” as used herein further includes compounds having one or more non-peptidic components in addition to a peptidic component.

For example, the term “peptide” encompasses compounds having modified amino acid(s) such as an amino acid analog, an amino acid derivative or an amino acid mimic. Examples of a modified amino acid include an α-hydroxy acid, a hydrazino amino acid, an amino-oxy acid, an aza-amino acid, a β-amino acid, a γ-amino acid, a D-amino acid, or an achiral amino acid.

For example, the term “peptide” includes not only compounds in which all amino acid residues are joined by peptide bonds (—C(O)NHR—) but also compounds in which one or more of the peptide bonds is replaced with an ester bond (—C(O)OR—) (such compounds being known as depsipeptides).

For example, the term “peptide” also encompasses compounds having one or more chemical modifications at its terminus. For example, a functional group may be attached at the N- and/or C-terminus of the compound. The functional group may include an amide group, an amine group, an acetyl group, a hydroxyl group, a thiol group, a malemide group or an acid group. It will also be appreciated that while the compound may be blocked at one or both termini, in some embodiments, the compounds may also have free or unblocked terminus (or termini).

For example, the term “peptide” also encompasses compounds having non-peptidic component attached to a peptidic component, for example a biomolecule attached to a N-terminal amino acid. The non-peptidic components may be a biotin, an alcohol or a saccharide.

The term “aliphatic amino acid” as used herein broadly refers to any amino acid which carbon chain is aliphatic in nature i.e. the carbon chain does not contain an aromatic ring. Non-limiting examples of an “aliphatic amino acid” include alanine, arginine, asparagine, aspartic acid, cysteine, glutamic acid, glutamine, glycine, isoleucine, leucine, lysine, methionine, proline, serine, threonine and valine.

The term “amphiphilic” as used herein in relation to a peptide refers to a peptide comprising both hydrophobic and hydrophilic moieties.

The term “self-assembly” as used herein in relation to a peptide refers to peptide that can organize into a higher order structure in response to conditions in the environment, such as when added in sufficient concentration to a liquid medium. The terms “self-assemble” and “self-assembling” are to be construed accordingly.

The term “micro” as used herein is to be interpreted broadly to include dimensions from about 1 micron to about 1000 microns.

The term “nano” as used herein is to be interpreted broadly to include dimensions less than about 1000 nm.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a peptide, such as an amphiphilic peptide, and related hydrogels, compositions, methods and uses are disclosed hereinafter.

In various embodiments, there is provided a peptide, e.g. amphiphilic peptide, comprising 3 or more amino acids or analogs thereof or derivatives thereof, wherein the peptide comprises a polar moiety/molecule/modification at the N-terminus and an amino acid, e.g. an aliphatic amino acid, or analogs thereof or derivatives thereof at the C-terminus. In some embodiments, the amphiphilic peptide comprises residues that are more hydrophilic at the N-terminal portion of the peptide, and residues that are more hydrophobic at the C-terminal portion of the peptide. In various embodiments, the residues at the C-terminal portion of the peptide is collectively more hydrophobic than the residues at the N-terminal portion of the peptide. Hence, the N-terminal portion may be collectively less hydrophobic than the C-terminal portion. For example, the N-terminal portion may be less hydrophobic than the C-terminal portion, or the N-terminal portion collectively may be hydrophilic, or the C-terminal portion collectively may not be more hydrophilic or the C-terminal portion collectively may not be less hydrophobic, wherein the collective degree of or relative hydrophobicity of the N-terminal portion and the C-terminal portion is such that the N-terminal portion is less hydrophobic than the C-terminal portion. In some embodiments, the average degree of hydrophobicity of the residues at the C-terminal portion of the peptide is more than the average degree of hydrophobicity of the residues at the C-terminal portion of the peptide. In some examples, the C-terminus half of the peptide is generally more hydrophobic or made to be more hydrophobic as compared to the N-terminus half.

In various embodiments therefore, there is provided an amphiphilic peptide having the formula (I):

XYZ (I)

wherein X is a polar moiety at the N-terminus; Y and Z each independently has between 1 to 4 residues of aliphatic amino acids or analogs or derivatives thereof, and wherein the average degree of hydrophobicity of the residues in block Z is more than the average degree of hydrophobicity of the residues in block Y. The amphiphilic peptide may comprise both hydrophobic and hydrophilic (or less hydrophobic) moieties. In various embodiments, the aliphatic amino acid is selected from the group consisting of alanine, arginine, asparagine, aspartic acid, cysteine, glutamic acid, glutamine, glycine, isoleucine, leucine, lysine, methionine, proline, serine, threonine and valine.

The amino acids in block Y and the amino acids in block Z may or may not be sequenced or arranged in order of increasing hydrophobicity towards the C-terminus. That is, each of the amino acid within the amino acid block Y and/or amino acid block Z may not be sequenced or arranged in order of increasing hydrophobicity. For example, when the amino acids in block Y comprise an amino acid sequence Y1-Y2-Y3 and the amino acids in block Z comprise an amino acid sequence Z1-Z2-Z3, the degree of hydrophobicity may be Y1≤ or ≥Y2≤ or ≥Y3≤Z1≤ or ≥Z2 or ≥Z3 so long as the entire Z block is > in hydrophobicity than Y block. In some examples, the degree of hydrophobicity may be: Y1<Y2<Y3<Z1<Z2<Z3, or Y1<Y2<Y3<Z1<Z2≤Z3, or Y1<Y2<Y3<Z1<Z2≥Z3 etc. so long as the entire Z block is > in hydrophobicity than Y block. The hydrophobicity of each block may be dependent on the hydrophobicity of the residues attached to the α-carbons in each block. In some examples, the C-terminus half of the hydrophobic block is generally more hydrophobic or made to be more hydrophobic compared to the N-terminus half.

In some embodiments, block Y and/or Z comprise or consist of aliphatic amino acids or analogs or derivatives thereof.

In some embodiments, the most hydrophobic aliphatic amino acids among the standard α-amino acids are generally isoleucine, leucine, valine. In some embodiments, the least hydrophobic aliphatic amino acids among the standard α-amino acids are generally arginine, aspartic acid, glutamic acid, asparagine and lysine. In some embodiments, alanine and glycine are more hydrophobic than serine. In some embodiments, alanine is more hydrophobic than glycine.

The molar moiety may or may not be a peptidic component i.e. the molar moiety can be non-peptidic. In various embodiments, the polar moiety at the N terminus is selected from the group consisting of a polar functional group, a polar amino acid, and a small polar biomolecule. In some embodiments, the polar functional group is selected from the group consisting of amine, acetyl, hydroxyl, thiol, maleimide, and acid. In some embodiments, the polar amino acid is selected from the group consisting of an amino acid with neutral side chain (or a neutral amino acid), an amino acid with a positive charged side chain (or a basic amino acid), and an amino acid with a negative charged side chain (or an acidic amino acid). The polar amino acid may not be an aliphatic amino acid. In some embodiments, the polar amino acid comprises an aromatic amino acid. In one embodiment, the polar amino acid comprises histidine. In some embodiments, the polar amino acid is selected from the group consisting of lysine, histidine, glycine, serine and aspartic acid. In some embodiments, the small polar biomolecule is selected from the group consisting of biotin, alcohol, and saccharide. It will be appreciated that other suitable polar functional group, polar amino acid, or small polar biomolecule may also be used while preserving a property, e.g. a self-assembling property, of the peptide.

In various embodiments, Y is an amino acid block having one or more amino acids independently selected from the group consisting of an amino acid with neutral side chain (or a neutral amino acid), a unique amino acid, an amino acid with a positive charged side chain (or a basic amino acid), an amino acid with a negative charged side chain (or an acidic amino acid), and an aliphatic amino acid with hydrophobic side chain. In some examples, Y may be acyclic.

In various embodiments, Z is an amino acid block having one or more amino acids independently selected from the group consisting of an amino acid with neutral side chain (or a neutral amino acid), a unique amino acid, an amino acid with a positive charged side chain (or a basic amino acid), an amino acid with a negative charged side chain (or an acidic amino acid), and an aliphatic amino acid with hydrophobic side chain. In some examples, Z may be acyclic.

In various embodiments, the amino acid with neutral side chain is selected from the group consisting of glycine (gly or G), asparagine (asn or N), cysteine (cys or C), glutamine (gin or Q), methionine (met or M), serine (ser or S), threonine (thr or T), and analogs thereof. In various embodiments, the unique amino acid is selected from the group consisting of glycine (gly or G), and analog thereof (for example glycolic acid (g)). In various embodiments, the amino acid with a positive charged side chain (or a basic amino acid) is selected from the group consisting of arginine (arg or R), histidine (his or H), lysine (lys or K), ornithine (Orn), 2,3-diaminopropionic acid (Dap), 2,4-diaminobutyric acid (Dab), and analogs thereof. In various embodiments, the amino acid with a negative charged side (or an acidic amino acid) is selected from the group consisting of aspartic acid (asp or D), glutamic acid (glu or E), and analogs thereof. In various embodiments, the aliphatic amino acid with hydrophobic side chain is selected from the group consisting of alanine (ala or A), isoleucine (ile or I), leucine (leu or L), valine (val or V), norleucine, homoallylglycine, homoproparylglycine, and analogs thereof (for example L-lactic acid (a)). In some embodiments, the aliphatic amino acid with hydrophobic side chain is selected from the group consisting of alanine (ala or A), isoleucine (ile or I), leucine (leu or L), and analogs thereof.

In some examples, where the aliphatic amino acid with hydrophobic side chain is alanine, the peptide has poor solubility in physiological solution. In some examples, where the aliphatic amino acid with hydrophobic side chain is isoleucine or leucine, the peptide has good solubility in physiological solution. In some examples, the peptide has good solubility in water.

In various embodiments, one or more peptide bond or peptide linkage in the peptide is modified to or replaced with an ester bond or an ester linkage. Thus, in some embodiments, the peptide is a depsipeptide. In some embodiments, the peptide, e.g. the amphiphilic peptide, comprises a depsipeptide analog. In some embodiments, the N-terminal portion of the amide backbone is replaced with an ester linkage.

In various embodiments, the peptide comprises modified amino acid(s). The modified amino acid may be an amino acid analog, an amino acid derivative or an amino acid mimic. In various embodiments, the modified amino acid comprises an α-hydroxy acid, a hydrazino amino acid, an amino-oxy acid, an aza-amino acid, a β-amino acid, a γ-amino acid, a D-amino acid, an achiral amino acid or combinations thereof.

In some embodiments, the peptide comprises an α-hydroxy acid analog. The α-hydroxy acid analog may be a glycolic acid (or g) and/or a L-lactic acid (or a). In some embodiments, the depsipeptide comprises an α-hydroxy acid analog. In some embodiments, the depsipeptide analog comprises an α-hydroxy acid analog.

In some embodiments, the peptide comprises a γ-amino acid analog.

In various embodiments, the peptide, or the aliphatic amino acid(s) comprises L-amino acid(s). In various embodiments, the peptide, or the aliphatic amino acid(s) comprises a mixture of D-amino acid(s) and L-amino acid(s). In various embodiments, the peptide, or the aliphatic amino acid(s) comprises D-amino acid(s). The incorporation of D-amino acids may prolong the in vivo stability or half-life of the peptide.

In some embodiments, the chirality of each of the residues in Y is different. In some embodiments, the chirality of each of the residues in Y is the same. In some embodiments, the chirality of each of the residues in Z is the same. In some examples, the peptides or building blocks assume a helical secondary structure when the chirality of each of the residues in Z is the same. In some embodiments, each of the residues in block Y and/or block Z are exclusively D-amino acid residues or L-amino acid residues. The chirality of Y and/or Z may be different or may be the same e.g. each of the residues in block Y may be D-amino acid residues while each of the residues in block Z may be L-amino acid residues, or vice versa, or each of the residues in block Y and Z may be D-amino acid residues etc.

In various embodiments, Z comprises a residue of an aliphatic amino acid with hydrophobic side chain, or analogs or derivatives thereof.

In some embodiments, the N-terminus of the peptide is modified. For example, the N-terminus may be modified with a protecting group. For example, a functional group may be attached at the N-terminus. The functional group may include an amine group, an acetyl group, a hydroxyl group, a thiol group, a malemide group or an acid group. In some embodiments, the N-terminus is acetylated.

In some embodiments, the N-terminus is not modified. In some embodiments, the N-terminus is free or unblocked. In some embodiments, the N-terminus comprises a free amine.

In various embodiments, the C-terminus is modified. For example, a functional group may be attached at the C-terminus. For example, the C-terminus may be modified to avoid zwitterion formation. In some embodiments, the C-terminus is amidated.

In some embodiments, the C-terminus is not modified. In some embodiments, the C-terminus is free or unblocked.

In some embodiments, the N-terminus and C-terminus are modified. In some embodiments, the N-terminus is acetylated (Ac) and/or C-terminus is amidated (NH₂).

In various embodiments, the peptide comprises a short peptide. In various embodiments, the peptide comprises an ultrashort peptide. In some embodiments, the peptide is no more than about 10 residues, no more than about 9 residues, no more than about 8 residues, or no more than about 7 residues in length. In some embodiments, the peptide is at least about 3 residues in length. In some embodiments, the peptide is from about 3 to about 8 residues in length, or about 3 to about 7 residues in length, or about 3 to about 6 residues in length. In some embodiments, the peptide comprises an ultrashort peptide that is from between about 3 to about 7 residues in length. The ultrashort peptide may be about 3 residues, about 4 residues, about 5 residues, about 6 residues or about 7 residues in length. In one embodiment, the peptide, e.g. the amphiphilic peptide, is no more than 7 residues in length.

In some examples, the peptide is selected from the group consisting of Ac-KgAVLI-NH₂, Ac-KGaVLI-NH₂, KgAVLI-NH₂, KGaVLI-NH₂, Ac-KgAVIL-NH₂, Ac-KGaVIL-NH₂, KgAVIL-NH₂, KGaVIL-NH₂, Ac-SgAVLI-NH₂, Ac-SGaVLI-NH₂, SgAVLI-NH₂, SGaVLI-NH₂, Ac-SgAVIA-NH₂, Ac-SGaVIA-NH₂, SgAVIA-NH₂, and SGaVIA-NH₂.

In some embodiments, X is an amino acid with a positive charged side chain or a basic amino acid (or analogs or derivatives thereof), and Y and Z are independently selected from the group consisting of a unique amino acid and an aliphatic amino acid with hydrophobic side chain (or analogs or derivatives thereof).

In some examples, the peptide comprises the sequence selected from the group consisting of KVI, KGAVLI, KGAVIL, KGAVIA, RVI, RGAVLI, RGAVIL, RGAVIA, HVI, HGAVLI, HGAVIL, HGAVIA, OrnVI, OrnGAVLI, OrnGAVIL, OrnGAVIA, DapVI, DapGAVLI, DapGAVIL, DapGAVIA, DabVI, DabGAVLI, DabGAVIL, DabGAVIA, KgAVLI, KGaVLI, KgAVIL, and KGaVIL.

In some examples, the peptide is selected from the group consisting of KGAVLI-NH₂, KGAVIL-NH₂, KgAVLI-NH₂, KGaVLI-NH₂, KgAVIL-NH₂, and KGaVIL-NH₂.

In some examples, the peptide is selected from the group consisting of Ac-KVI-NH₂, Ac-KGAVLI-NH₂, Ac-KGAVIL-NH₂, Ac-KGAVIA-NH₂, Ac-RVI-NH₂, Ac-RGAVLI-NH₂, Ac-RGAVIL-NH₂, Ac-RGAVIA-NH₂, Ac-HVI-NH₂, Ac-HGAVLI-NH₂, Ac-HGAVIL-NH₂, Ac-HGAVIA-NH₂, Ac-OrnVI-NH₂, Ac-OrnGAVLI-NH₂, Ac-OrnGAVIL-NH₂, Ac-OrnGAVIA-NH₂, Ac-DapVI-NH₂, Ac-DapGAVLI-NH₂, Ac-DapGAVIL-NH₂, Ac-DapGAVIA-NH₂, Ac-DabVI-NH₂, Ac-DabGAVLI-NH₂, Ac-DabGAVIL-NH₂, Ac-DabGAVIA-NH₂, KGAVLI-NH₂, KGAVIL-NH₂, Ac-KgAVLI-NH₂, Ac-KGaVLI-NH₂, KgAVLI-NH₂, KGaVLI-NH₂, Ac-KgAVIL-NH₂, Ac-KGaVIL-NH₂, KgAVIL-NH₂, and KGaVIL-NH₂.

In some embodiments, X is an amino acid with a neutral side chain (or analogs or derivatives thereof), and Y and Z are independently selected from the group consisting of a unique amino acid and an aliphatic amino acid with hydrophobic side chain (or analogs or derivatives thereof).

In some examples, the peptide comprises the sequence selected from the group consisting of GAVLI, SGAVLI, SGAVIL, SGAVIA, TGAVLI, TGAVIL, TGAVIA, SGAVLI, SGAVIA, SgAVLI, SGaVLI, SgAVIA, and SGaVIA.

In some examples, the peptide is selected from the group consisting of SGAVLI-NH₂, SGAVIA-NH₂, SgAVLI-NH₂, SGaVLI-NH₂, SgAVIA-NH₂, and SGaVIA-NH₂.

In some examples, the peptide is selected from the group consisting of Ac-GAVLI-NH₂, Ac-SGAVLI-NH₂, Ac-SGAVIL-NH₂, Ac-SGAVIA-NH₂, Ac-TGAVLI-NH₂, Ac-TGAVIL-NH₂, Ac-TGAVIA-NH₂, SGAVLI-NH₂, SGAVIA-NH₂, Ac-SgAVLI-NH₂, Ac-SGaVLI-NH₂, SgAVLI-NH₂, SGaVLI-NH₂, Ac-SgAVIA-NH₂, Ac-SGaVIA-NH₂, SgAVIA-NH₂, SGaVIA-NH₂.

In some embodiments, X is an amino acid with a negative side chain or an acidic amino acid (or analogs or derivatives thereof), and Y and Z are independently selected from the group consisting of a unique amino acid and an aliphatic amino acid with hydrophobic side chain (or analogs or derivatives thereof).

In some examples, the peptide comprises the sequence selected from the group consisting of DVI, DGAVLI, DGAVIL, EVI, EGAVLI, EGAVIL, and EGAVIA.

In some examples, the peptide is selected from the group consisting of Ac-DVI-NH₂, Ac-DGAVLI-NH₂, Ac-DGAVIL-NH₂, Ac-EVI-NH₂, Ac-EGAVLI-NH₂, Ac-EGAVIL-NH₂, and Ac-EGAVIA-NH₂.

In various embodiments, the peptide, e.g. the amphiphilic peptide, is selected from the group consisting of:

KVI, KGAVLI, KGAVIL, KGAVIA, RVI, RGAVLI, RGAVIL, RGAVIA, HVI, HGAVLI, HGAVIL, HGAVIA, OrnVI, OrnGAVLI, OrnGAVIL, OrnGAVIA, DapVI, DapGAVLI, DapGAVIL, DapGAVIA, DabVI, DabGAVLI, DabGAVIL, DabGAVIA, KgAVLI, KGaVLI, KgAVIL, KGaVIL;

KGAVLI-NH₂, KGAVIL-NH₂, KgAVLI-NH₂, KGaVLI-NH₂, KgAVIL-NH₂, and KGaVIL-NH₂,

Ac-KVI-NH₂, Ac-KGAVLI-NH₂, Ac-KGAVIL-NH₂, Ac-KGAVIA-NH₂, Ac-RVI-NH₂, Ac-RGAVLI-NH₂, Ac-RGAVIL-NH₂, Ac-RGAVIA-NH₂, Ac-HVI-NH₂, Ac-HGAVLI-NH₂, Ac-HGAVIL-NH₂, Ac-HGAVIA-NH₂, Ac-OrnVI-NH₂, Ac-OrnGAVLI-NH₂, Ac-OrnGAVIL-NH₂, Ac-OrnGAVIA-NH₂, Ac-DapVI-NH₂, Ac-DapGAVLI-NH₂, Ac-DapGAVIL-NH₂, Ac-DapGAVIA-NH₂, Ac-DabVI-NH₂, Ac-DabGAVLI-NH₂, Ac-DabGAVIL-NH₂, Ac-DabGAVIA-NH₂, Ac-KgAVLI-NH₂, Ac-KGaVLI-NH₂, Ac-KgAVIL-NH₂, Ac-KGaVIL-NH₂;

GAVLI, SGAVLI, SGAVIL, SGAVIA, TGAVLI, TGAVIL, TGAVIA, SGAVLI, SGAVIA, SgAVLI, SGaVLI, SgAVIA, and SGaVIA;

SGAVLI-NH₂, SGAVIA-NH₂, SgAVLI-NH₂, SGaVLI-NH₂, SgAVIA-NH₂, and SGaVIA-NH₂,

Ac-GAVLI-NH₂, Ac-SGAVLI-NH₂, Ac-SGAVIL-NH₂, Ac-SGAVIA-NH₂, Ac-TGAVLI-NH₂, Ac-TGAVIL-NH₂, Ac-TGAVIA-NH₂, Ac-SgAVLI-NH₂, Ac-SGaVLI-NH₂, Ac-SgAVIA-NH₂, Ac-SGaVIA-NH₂;

DVI, DGAVLI, DGAVIL, EVI, EGAVLI, EGAVIL, and EGAVIA; and

Ac-DVI-NH₂, Ac-DGAVLI-NH₂, Ac-DGAVIL-NH₂, Ac-EVI-NH₂, Ac-EGAVLI-NH₂, Ac-EGAVIL-NH₂, and Ac-EGAVIA-NH₂, wherein Orn=ornithine, Dap=2,3-diaminopropionic acid, Dab=2,4-diaminobutyric acid, g=glycolic acid and a=L-lactic acid.

In various embodiments, the peptide comprises a peptidic/sequence motif. The motif may drive molecular self-assembly/self-organisation into nanofibrous scaffolds via α-helical intermediates/fibers. In some embodiments, the motif comprises a short series of aliphatic amino acids (or analogs or derivatives thereof) arranged, generally, in increasing hydrophobicity from N- to C-terminus.

In some embodiments, the peptide is capable of self-assembling into an α-helical secondary structure or may be capable of transitioning from a random coil to a α-helical structure. In some embodiments, the peptide is capable or further capable of self-assembling into a β-type structure. In some embodiments, the peptide is capable of transitioning from a random coil to an α-helical structure and subsequently to a β-type structure, for example, when the peptide is provided with increasing concentration. In some examples, the peptide may transform into (a) random coil to α-helical; and (b) α-helical to β-type structures at critical concentration. As will be appreciated, the critical concentration would vary depending on the sequence of the peptide. In one embodiment, the peptide, e.g. the amphiphilic peptide, is capable of self-assembling into a hydrogel.

The peptide may also be capable of forming aggregates. For example, the peptide may be capable of gelation or assembling into hydrogels, such as nanofibrous hydrogels, on exposure to a stimuli. For example, the stimuli may be a solution including, but not limited to, an aqueous medium, a salt solution, a buffered salt solution, a buffered saline solution, or a phosphate buffered saline solution, and the like (e.g. other liquid medium). The mechanism of self-assembly into nanofibers in aqueous conditions and polar solvents may be unique. The resulting scaffold or hydrogel may be biomimetic due to its resemblance to extracellular matrix. Advantageously, this makes the material desirable or ideal for cell culture and tissue regeneration.

In some embodiments, the peptide is both soluble under specified condition and capable of self-assembling into a hydrogel. Based on the teaching provided herein, it would not be beyond the skill of a person in the art to determine the residues required to have a balance between having sufficiently hydrophobic residues at the C-terminus vs polar moiety at the N-terminus that would still allow for suitable solubility in desired solution, whilst at the same time maintaining the ability to self-assemble at low concentration.

In various embodiments, there is provided a composition or hydrogel comprising the peptide, e.g. the amphiphilic peptide, as described herein. In some embodiments, the hydrogel comprises a nanofibrous hydrogel. In some embodiments, the hydrogel comprises a biomimetic hydrogel. In some embodiments, the hydrogel comprises a biomimetic nanofibrous hydrogel.

In some embodiments, the peptide, the composition or the hydrogel is capable of forming scaffolds (such as a nanofibrous scaffolds). The scaffolds may entrap one or more substances. The substances that may be entrapped by the peptide, the composition or the hydrogel are not particularly limited. In some examples, the peptide, the composition or the hydrogel is capable of entrapping at least one substance selected from the group consisting of water, other polar solvents, a microorganism, a virus particle, a peptide, a peptoid, a protein, a nucleic acid, an oligosaccharide, a polysaccharide, a vitamin, an inorganic molecule, a polymer (e.g. a synthetic polymer), an organic molecule (e.g. a small organic molecule), a biologically active ligand, or a pharmaceutically active compound. In some examples, the peptide, the composition or the hydrogel comprises encapsulated biological cells, cellular spheroids, organoids or 3D organotypic constructs. In some examples, the macromolecular assembly entraps a high proportion of water (e.g. during self-assembly of the peptides), forming rigid hydrogels with storage moduli exceeding 1 kPa.

In some embodiments, the peptide, the composition or the hydrogel comprises one or more agents associated with or capable of inducing cell proliferation/differentiation/growth. Examples of such agents include but are not limited to, Activin A, VEGF, BMP2, EGF, BDNF, FGF4, Wnt3A, or TGF-β. As may appreciated, depending on the cell type and the intended goal, other suitable growth factors, transcription factors, medium etc. may also be used.

In some embodiments, the peptide, the composition or the hydrogel has high permeability for oxygen, nutrients, and other water-soluble metabolites.

In some embodiments, the peptide, the composition or the hydrogel is biocompatible. Embodiments of the peptide, the composition or the hydrogel can therefore be suitably used in a variety of biomedical applications, such as to encapsulate cells for 3D cell culture. Further, embodiments of the composition or the hydrogel are also stable and not easily dissociated upon gelation. Advantageously, cells or other materials encapsulated by embodiments of the composition or the hydrogel cannot escape.

Pluripotent stem cells show a tendency to aggregate into one single colony in a reproducible and consistent fashion in some embodiments of the peptide, the composition or the hydrogel. In some examples, when single cells are seeded on or in the hydrogel, the hydrogel is more capable of promoting cell migration and/or generating single colony as compared to a hydrogel composed of peptides having sequences that are inverted from the sequences of said amphiphilic peptide. Advantageously, the stability and consistent and reproducible production of single colony enabled by embodiments of the hydrogel make them desirable matrices for certain biological applications such as for stem cell differentiation into organoid or for bioprinting stem cell arrays for deriving organoid cultures which can be applied towards high throughput screening. Advantageously, enhanced cell migration enabled by embodiments of the hydrogel can also be exploited to deliver cells to regenerate damaged tissue.

In some embodiments, the peptide, the composition or the hydrogel is biodegradable.

In some embodiments, the peptide, the composition or the hydrogel is xenofree. In some examples, the xenofree peptide, the composition or the hydrogel is prepared using solid-phase peptide synthesis. Advantageously, the synthesis process is facile, customizable and scalable.

In some embodiments, the peptide, the composition or the hydrogel is injectable to a subject in need thereof.

In some embodiments, the peptide, the composition or the hydrogel demonstrates stimuli-responsive gelation. In some embodiments, the peptide, the composition or the hydrogel demonstrates salt and/or pH-responsive gelation. In some examples, instantaneous gelation can be obtained upon exposure to a physiologically compatible salt solution. In some examples, where the peptide comprises acidic peptide, subjecting the peptide to a solution having a pH of no more than about 7 (or less than about 7) induces or enhances gelation. In some examples, where the peptide comprises basic peptide, subjecting the peptide to a solution having a pH of at least 6.5 (or more than 6.5) induces or enhances gelation. In some examples, increasing the surrounding temperature, e.g. a temperature of the solvent carrying the peptide, from about 4° C. to about 37° C. induces or enhances gelation. Advantageously, this property can be exploited for applications such as bioprinting, drug screening (e.g. biofabrication of in vitro organotypic models), and developing injectable scaffolds for regenerative medicine (e.g. to fill tissue defects in vivo and to expand therapeutic stem cells ex vivo).

In some embodiments, the peptide, the composition or the hydrogel is thixotrophic. For example, the peptide, the composition or the hydrogel may have time-dependent shear thinning property or is capable of forming thick or viscous hydrogel under static condition or is capable of thinning or less viscous under shaken, agitated, sheared or stressed conditions. In some examples, the peptide, the composition or the hydrogel is in a gel-like state when standing and changes to a fluid-like state when shaken or agitated. In some examples, the peptide, the composition or the hydrogel is capable of changing from a fluid-like state to a gel-like state when left to stand for a period of time not exceeding about 30 minutes, about 25 minutes, about 20 minutes, about 15 minutes, about 10 minutes or about 5 minutes.

In some embodiments, the peptide, the composition or the hydrogel is comprised in at least a biosensing device, a medical device, a bioprinting device, an implant, a pharmaceutical composition, or a cosmetic composition.

In various embodiments, the peptide, e.g. the amphiphilic peptide, the composition or the hydrogel has one or more properties selected from the group consisting of: stable, biocompatible, biodegradable, biomimetic, xenofree, injectable, thixotrophic, substantially non-mutagenic, substantially resistant to enzymatic degradation, responsive to stimulus, responsive to change in pH, responsive to change in salt concentration, responsive to change in temperature, compatible with bioprinting and has a storage modulus of at least 1 kPa.

In various embodiments, there is provided a method of preparing a hydrogel, comprising providing a peptide as described herein in a condition suitable for inducing the aggregation of the peptide thereof. The condition suitable for aggregation of the peptide may include, but is not limited to, providing a stimuli such as change of a salt concentration, change of a pH, change of a temperature, providing a solution comprising salts, providing a solution having appropriate pH and increasing temperature. In some examples, where the peptide comprises acidic peptide, subjecting the peptide to a solution having a pH of no more than about 7 (or less than about 7) induces aggregation. In some examples, where the peptide comprises basic peptide, subjecting the peptide to a solution having a pH of at least 6.5 (or more than 6.5) induces aggregation. In some examples, increasing the surrounding temperature, e.g. a temperature of the solvent carrying the peptide, from about 4° C. to about 37° C. induces aggregation.

In various embodiments, there is provided a peptide, e.g. an amphiphilic peptide, a composition or a hydrogel for use in therapy. In various embodiments, there is provided an amphiphilic peptide, a composition or a hydrogel for use in surgery.

In various embodiments, there is provided a composition comprising an effective amount of the peptide for use in therapy in a subject in need thereof, optionally wherein the composition further comprises a suitable carrier, adjuvant, diluent and/or excipient.

In various embodiments, there is provided a pharmaceutical and/or cosmetic composition and/or a biomedical device and/or electronic devise comprising the peptide as described herein. The pharmaceutical composition may further comprise a pharmaceutically acceptable carrier and/or pharmaceutically active compound.

In various embodiments, there is provided a kit comprising the peptide or the composition or the hydrogel as described herein.

In various embodiments, there is provided a method of tissue regeneration comprising: providing the peptide, or the composition, or the hydrogel as described herein, contacting the peptide, or the composition, or the hydrogel to a cell of the tissue or organoid, and culturing the cell (under suitable condition) to form the regenerated tissue or organoid or part thereof in the presence of the peptide, or the composition, or the hydrogel. The tissue regeneration may be at least a partial regeneration, reconstruction, repair, replacement, restoration, or regrowth of a tissue, organ, or other body structure, or portion thereof, typically following loss, damage, or degeneration. In various embodiments, there is provided a peptide, a composition or a hydrogel for use in the manufacture or repair of tissue in a subject in need thereof. In various embodiments, there is provided use of the peptide or the composition or the hydrogel in the manufacture of a medicament for tissue regeneration.

In various embodiments, there is provided a method of treating a subject in need of tissue regeneration, the method comprising administering the peptide, e.g. the amphiphilic peptide, the composition or the hydrogel as described herein into the subject in need thereof. The method may be performed in vitro or ex vivo or in vivo. For example, where the method is performed in vivo, the peptide, or the composition, or the hydrogel is provided in a body of a subject where tissue regeneration is desired.

In various embodiments, there is provided a method of cell, tissue or organoid culture, the method comprising: culturing the cell, the tissue or the organoid in contact with or on the amphiphilic peptide, the composition, or the hydrogel. The culture may be one or more of a proliferation culture, a differentiation culture, a tissue culture, an organoid culture, a tissue regeneration culture, an organoid regeneration culture, a cell maintenance culture, a tissue maintenance culture, an organoid maintenance culture and the like. As may be appreciated, depending on the intended goal of the culture, suitable conditions, materials and agents may also be employed in the method.

In some embodiments, the cell comprises a stem cell e.g. a pluripotent stem cell or multipotent stem cell. In some embodiments, the method comprises a method of differentiating a stem cell. In some embodiments, there is provided a hydrogel when used in culturing stem cell, tissue or organoid.

In various embodiments, there is provided a method of culturing/differentiating/proliferating/encapsulating a cell or organoid (such as a stem cell or a pluripotent stem cell) comprising: providing the peptide, or the composition, or the hydrogel, and culturing or encapsulating the cell or organoid in the peptide, the composition, or the hydrogel (in a suitable condition thereof).

In various embodiments, there is provided a method of testing a compound (or drug screening) comprising: culturing a cell or a plurality of cells or an organoid in or on the peptide, or the composition, or the hydrogel, contacting the cell or the plurality of cells or the organoid to the compound, and detecting any morphological/physiological/gene or protein expression changes in the cell.

In various embodiments, there is provided a peptide or a composition or a hydrogel for use as bio-ink or bio-resin for the 3-dimensional biofabrication or 3-dimensional bioprinting of a biological construct. Optionally, the biological construct may be an animal tissue or organ or part thereof, optionally the biological construct may be a scaffold containing cells which may be porous or non-porous).

In various embodiments, there is provided a method of producing a peptide capable of self-assembly into an α-helical (alpha-helical) structure, the method comprising: identifying the sequence of a first peptide that has an α-helical structure, producing a second peptide having a sequence that is inverted from the sequence of the first peptide. In some embodiments, the second peptide comprises amino acids sequenced or arranged from the N-terminus to C-terminus in increasing hydrophobicity.

In various embodiments, there is provided a method or peptide or a hydrogel or a composition as described herein.

### BRIEF DESCRIPTION OF FIGURES

FIG. 1. Second generation self-assembling ultrashort peptide motif. (a) Sequence and structure of the first (exemplified by Ac-ILVAGK-NH₂) and second (exemplified by Ac-KGAVLI-NH₂) generation amphiphilic motifs. While the directionality of the peptide backbone is reversed, both peptide motifs consist of a chain of aliphatic amino acids with increasing hydrophilicity, terminating with a polar residue such as lysine. (b) During self-assembly, the inverted sequence transitions from random coil (dotted line) to alpha-helical (dashed line) to beta secondary structures (solid line) with increasing peptide concentration. This suggests that the peptides form intermolecular helical pairs which subsequently stack into beta-turn fibrils that aggregate into nanofibers and sheets visible under (c) field emission scanning electron microscopy. The resulting macromolecular scaffolds entrap water to form clear hydrogels. (d) The peptides demonstrate salt-enhanced gelation, (e) forming stiff hydrogels with storage moduli of up to 20 kPa in buffered saline solutions.

FIG. 2. Self-assembly of the inverted tripeptide sequence. Peptide conformational changes from random coil (dotted line) to α-helical intermediates (dashed line) to β-fibrils (solid line) as concentration increases. The peptide dimers subsequently stack in fibrils that aggregate into nanofibers and sheets, which entrap water to form hydrogels. The nanofibrous architecture, as observed using field emission scanning microscopy, resembles extracellular matrix. The fibers extend into the millimeter range and readily condense into sheets. The fibers form interconnected three-dimensional scaffolds which are porous.

FIG. 3. Second generation ultrashort peptides with (a) free N-terminus and (b) glycine and histidine as the polar moieties are capable of macromolecular assembly into gels.

FIG. 4. Self-assembly of Depsipeptides. (a) Like their parent peptide sequences, depsipeptides also self-assemble into hydrogels in aqueous conditions. Gelation can be enhanced by increasing pH, salt and depsipeptide concentration. (b) Depsipeptides undergo the same secondary structure transitions from random coil to α-helical and subsequently to β-type structures with increasing concentration.

FIG. 5. Ultrashort tripeptides with amidated aspartic acid at the C-terminus exhibit self-assembly into nanofibrous hydrogels following dissolution in water and PBS. (a), (b) Nanofibrous microarchitecture of Ac-DVI-NH₂ as revealed by field emission scanning microscopy. (c) Thixotropic behavior of Ac-DVI-NH₂ hydrogels is reflected by recovery of the intact gel following disturbance by vortexing. Left: Ac-DVI-NH₂ in a gel state before vortexing. Middle: Ac-DVI-NH₂ changed to a fluid state immediately after vortexing. Right: Ac-DVI-NH₂ returned to gel state after standing for 10 minutes after vortexing.

FIG. 6. 3D culture of human pluripotent stem cells. (a) H1 human embryonic stem cells were cultured in Ac-KGAVLI-NH₂ hydrogel droplets under the following conditions: (i) in a proliferation media at a cell concentration of 2×10⁶ cells/mL; (ii) in a proliferation media at a cell concentration of 5×10⁶ cells/mL; and (iii) in an endoderm differentiation media at a cell concentration of 5×10⁶ cells/mL. The cells migrated and proliferated within the gel to cluster around a central nucleus at seeding densities exceeding 5×10⁶ cells/m L, while several colonies were observed to be formed at the lower seeding density of 2×10⁶ cells/m L. This behavior is independent of media formulation, and in over 95% of the colonies for H1 and H9 cells, only one central stem cell colony was obtained. (b) The H1 human embryonic stem cells retained their pluripotency, as evident from the staining of Oct4 and Tral-60 biomarkers, when cultured in mTESR media. (c) The Ac-KGAVLI-NH₂ peptide was non-mutagenic, as evident from the lack of chromosomal aberrations after 5 passages of 3D culture. The hydrogel can thus be applied towards bioprinting of consistent stem cell colonies for high-throughput screening applications.

FIG. 7. 3D “one-pot” endoderm organoid derivation. (a) Encapsulated H1 embryonic stem cells were directly differentiated into definitive endoderm and subsequently hindgut spheroids without going through intermediate 2D culture steps. (b) Definitive endoderm differentiation was verified by confocal staining of Sox17 and FoxA2 biomarkers. 90% of the cells express Sox17, as determined by flow cytometry. (c) Similarly, expression of hindgut biomarker Cdx2 was observed on day 9, following further differentiation.

FIG. 8. 3D differentiation of H9 embryonic stem cells encapsulated in 8 mg/mL Ac-KGAVLI-NH2 hydrogel droplets. (a) Definitive endoderm differentiation was verified by confocal staining of Sox17 and FoxA2 biomarkers. 90% of the cells express Sox17, as determined by flow cytometry. (b) Similarly, expression of hindgut biomarker Cdx2 was observed on day 9, following further differentiation.

FIG. 9. Porcine wound healing study of peptide hydrogel wound dressings; Three peptide hydrogels applied using a polydimethylsiloxane mould to 8 cm×8 cm excisional wound. Wound dressing changes carried out weekly for 6 weeks with application of fresh hydrogel. P1: Ac-HGAVLI-NH2; P2: SGAVLI-NH2; P3: Ac-KGAVLI-NH2; C: control

### EXAMPLES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments.

### Materials and Methods

Materials. All peptides used in this study were either synthesized by solid-phase peptide synthesis, purified using high-performance liquid chromatography mass spectrometry and lyophilized, or purchased from Bachem AG (Bubendorf, Switzerland). Human H1 and H9 embryonic stem cells were purchased from WiCell Research Institute (Madison, Wis.). Reagents for culture of human embryonic stem cells were purchased from Stem Cell Technologies (British Columbia, Canada). All other cell culture reagents were purchased from Life Technologies (Carlsbad, Calif.). For immunohistochemistry, the primary antibodies used were Ab19857 rabbit polyclonal IgG against Oct 4 (Abcam, Cambridge, Mass.), SC-21705 mouse monoclonal IgM against Tra-I-60 (Santa Cruz Biotechnology Inc, Dallas, Tex.), AF1924-SP goat polyclonal IgG against human Sox17 (R&D Systems, USA), MAB2400-SP rabbit monoclonal IgG against human FoxA2 (R&D Systems, USA) and, MAB3665-SP mouse monoclonal IgG against human Cdx2 (R&D Systems, USA). 4′,6-diamidino-2-phenylindole (DAPI) (Invitrogen, Carlsbad, Calif.) was used to stain the actin cell nuclei.

Preparation of Hydrogels.

Lyophilized peptide powder was dissolved in milliQ water and mixed for 30 seconds by vortexing to obtain a homogenous solution. 10% volume of 10-times phosphate-buffered saline was subsequently added and mixed by pipetting. Gelation occurred between seconds (PBS) to overnight (water), depending on the peptide concentration and solution used.

Circular Dichroism Spectroscopy.

CD spectra were collected with a Jasco CD spectrophotometer fitted with a temperature controller, using quartz suprasil cuvettes with an optical path length of 1 mm. All samples were prepared in milIQ water and equilibrated for an hour at room temperature before measurement. Data acquisition was performed for wavelength range from 190-260 nm with a spectral bandwidth of 1.0 nm. All spectra were baseline-corrected using milliQ water as baseline. The mean residue ellipticity (MRE) was calculated as follows:

[θ]=θ/(10·N·c·l) where 8 represents the ellipticity in millidegrees, N the number of amino acid residues, c the molar concentration in mol·L-1, and I the cell path length in cm.

Field Emission Scanning Electron Microscopy.

Hydrogel samples were flash frozen in liquid nitrogen and subsequently freeze-dried. Lyophilized samples were sputtered with platinum in a JEOL JFC-1600 High Resolution Sputter Coater. The coated sample was then examined with a JEOL JSM-7400F FESEM system using an accelerating voltage of 2-5 kV.

Rheology.

Hydrogel samples were prepared in polydimethysiloxane moulds to obtain approximately 1 mm thick, 8 mm diameter discs. Dynamic strain and oscillatory frequency sweep experiments were carried out using the ARES-G2 Rheometer (TA Instruments, Piscataway, N.J.) with 8 mm titanium parallel plate geometry. The readings of 3 samples were averaged for each condition.

3D Encapsulation of Stem Cells.

H1 embryonic stem cells cultured on Matrigel were dissociated into single cells using TrypLE Express, and re-suspended in 50% mTESR in PBS at an approximate concentration of 4×10⁶, 10⁷ or 1.6×10⁷ cells/mL. 0.5 μL of cells was injected into a droplet of 2 μL 10 mg/mL peptide solution. Warmed culture media (mTESR) containing ROCK inhibitor Y-27632 was added for the first day and replaced by either mTESR or endoderm differentiation media subsequently. The endoderm differentiation protocol was adapted from Spence et al (2011). Briefly, the encapsulated cells were exposed to RPMI media containing 100 ng/mL Activin A, 2 mM glutamax, 1% penicillin-streptomycin and increasing concentrations of defined fetal bovine serum. After 3 days, the hydrogel droplets were washed with RPMI and incubated in hindgut differentiation media (RPMI media containing 1% defined fetal bovine serum, 1% penicillin-streptomycin, 2 mM glutamax, 500 ng/mL FGF4 and 500 ng/mL Wnt3A.

Immunohistochemistry and Confocal Microscopy Imaging.

Cell samples were fixed in 4% paraformaldehyde for 15 minutes and permeabilized in 0.01% Triton-X for 10 minutes. The encapsulated human embryonic stem cells were incubated at 4° C. overnight in 5% Bovine-Serum Albumin containing primary antibodies. The corresponding secondary antibodies and DAPI were applied for 90 minutes before the samples were imaged. Confocal microscopy was performed using a Zeiss LSM 510 microscope at the Institute of Medical Biology Microscopy Unit (A*STAR, Singapore).

### EXAMPLES

Before the present disclosure, the effect of sequence inversion has never been reported or systematically studied. Because of the strict rules governing the design of self-assembling helical peptides, the directionality change in peptide backbone was expected to disrupt intra-helical hydrogen bonding and thus macromolecular organization. Beta-sheet peptide self-assembly was expected to be affected by a smaller extent due to their characteristic motif of alternating hydrophilic-hydrophobic residues, as well as the planar nature of intermolecular interactions. Almost palindromic beta-sheet sequences have been described.

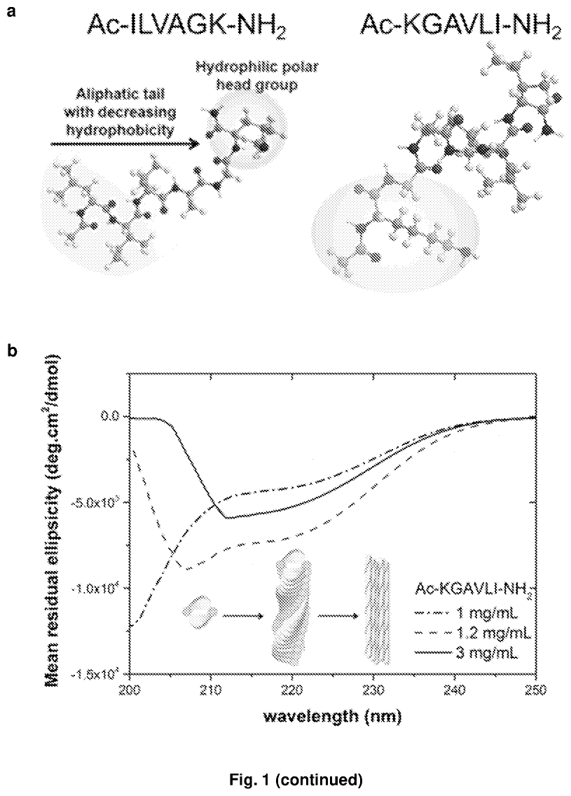

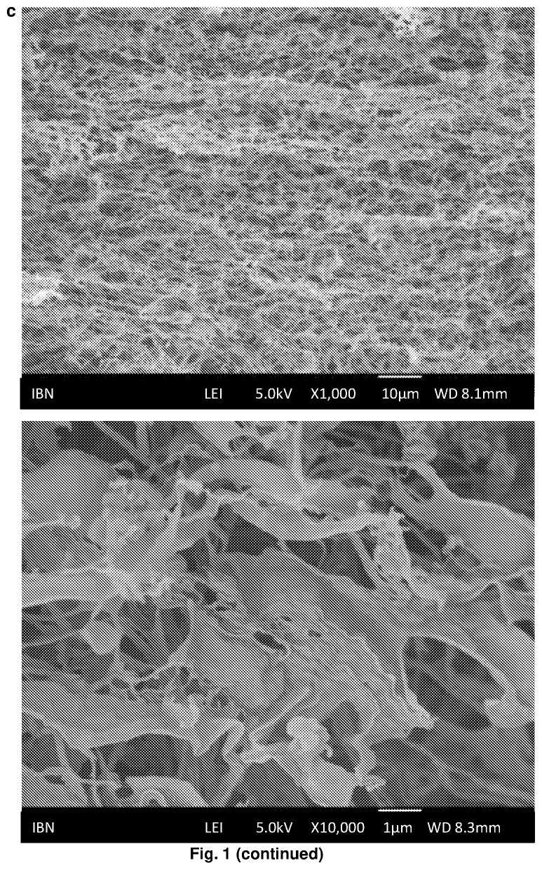

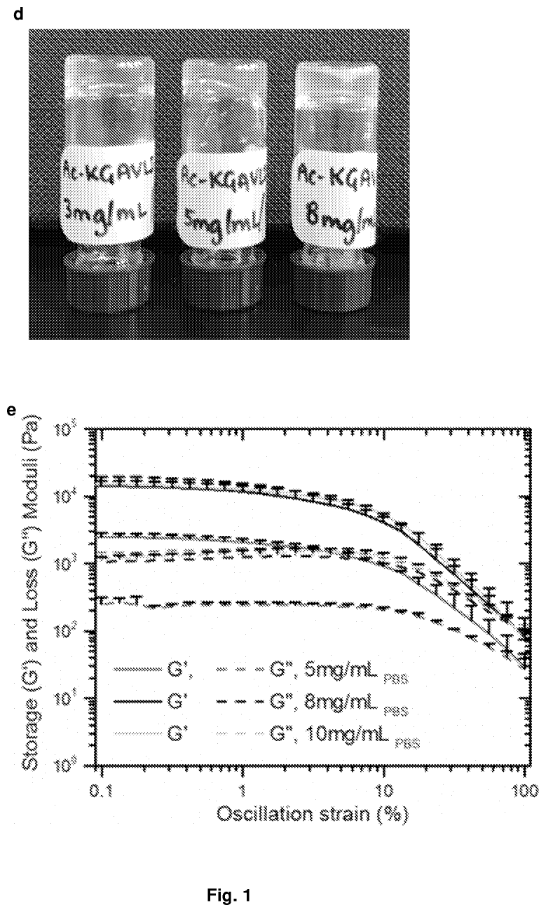

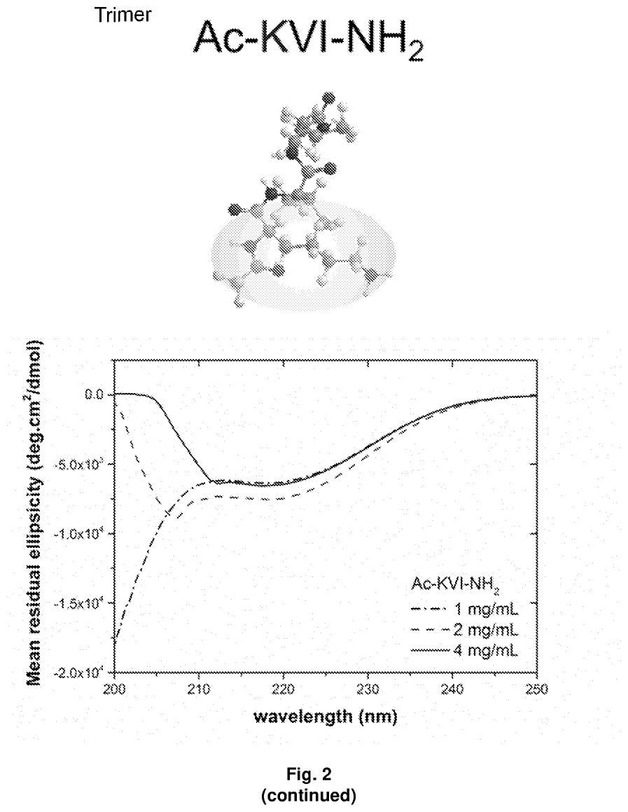

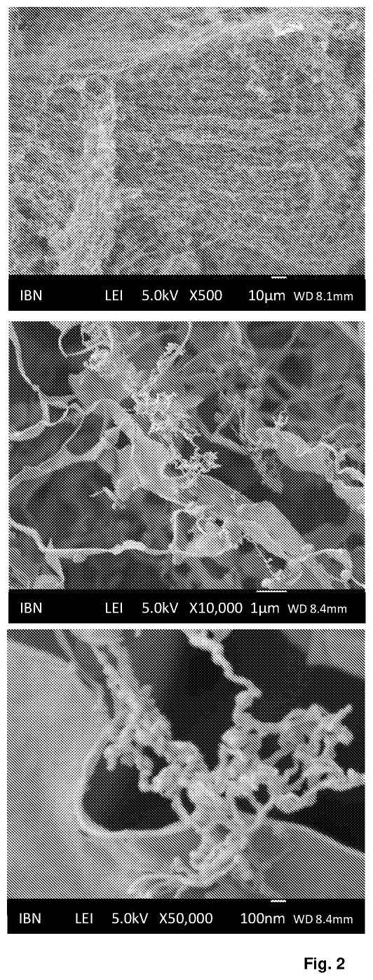

Surprisingly, the self-assembly of ultrashort peptides into helical fibers was found to be unaffected by sequence inversion and the consequent reversal in peptide backbone direction. Trimeric and hexameric inverted sequences, exemplified by Ac-KVI-NH₂ and Ac-KGAVLI-NH₂ (FIG. 1a, FIG. 2), are observed to undergo the same secondary structure transitions as their parent sequences. Circular dichroism spectra at increasing concentrations of peptide are surrogate snapshots of the structural transitions that occur during macromolecular assembly. At low concentrations, the monomers adopt a random coil confirmation with a slight positive n-π* transition near 217 nm and large negative transition around 190 nm (FIG. 1b). At higher concentrations, alpha-helices with their characteristic signature of a negative n-π* transition near 222 nm and split π-π* transition with a negative peak near 208 nm were observed. Further increases in concentration saw the development of beta-turn structures with negative bands at 218 nm. As the structural transition profiles are virtually identical to that of the original parent sequences of Ac-IVK-NH₂ and Ac-ILVAGK-NH₂, it is surmised that they follow the same self-assembly mechanism wherein the peptide monomers form anti-parallel pairs and subsequently stack to form beta-turn fibrils. It is postulated that since the turns within each helical fibril are not covalently linked, each succeeding pair of peptides can rotate laterally during fibril assembly to maximize intermolecular hydrogen bonding and hydrophobic interactions. In contrast, freedom of movement is restricted in the longer 28-mer coiled-coil and 30-mer collagen mimetic motifs. As such, there is no mention in published literature that the reverse sequences of heptad coiled-coils and collagen-mimetic peptides can self-assemble. The present experiment indicates that in the case of ultrashort peptides, the building block that dictates the macromolecular assembly is the amphiphilic motif, regardless of peptide backbone orientation.

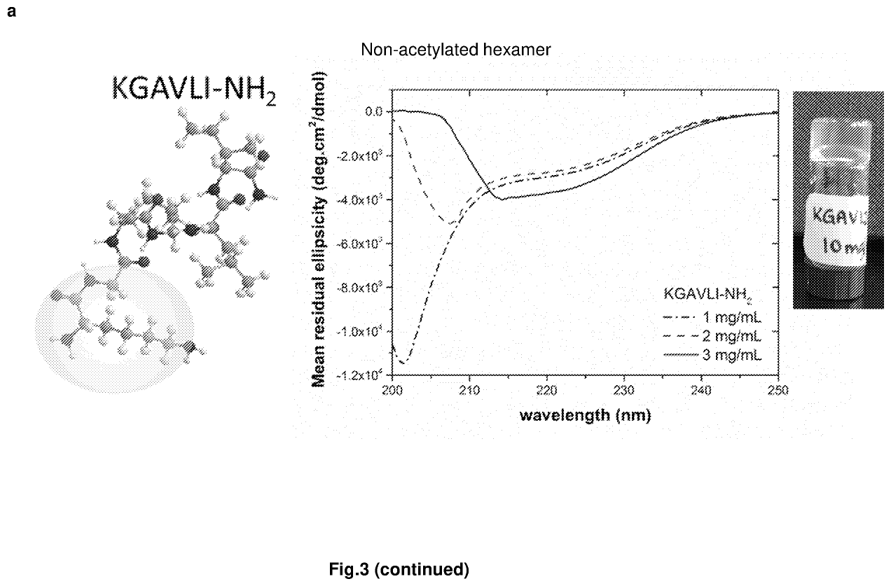

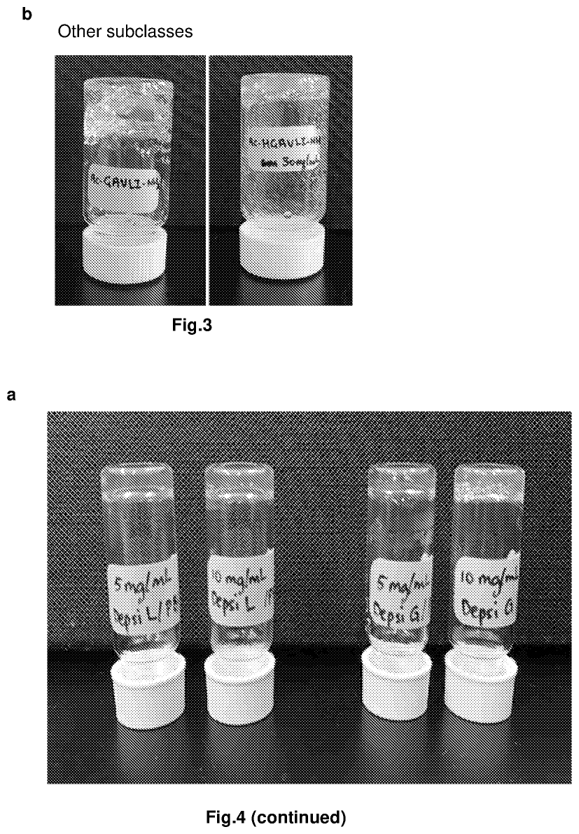

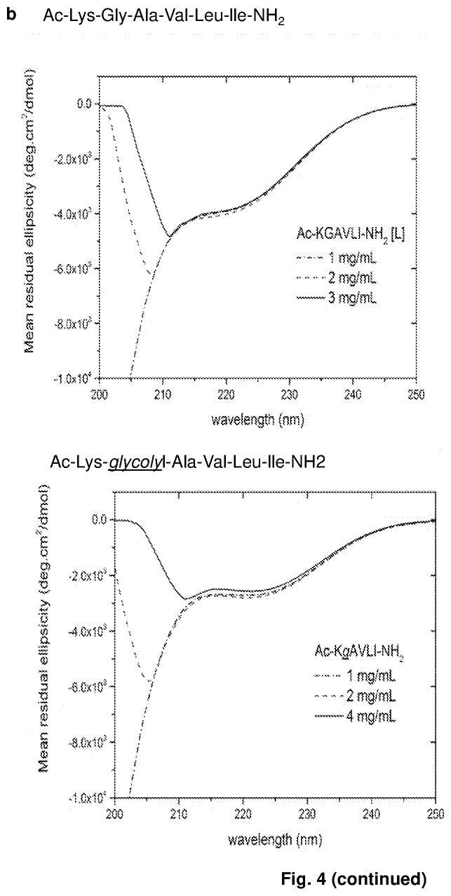

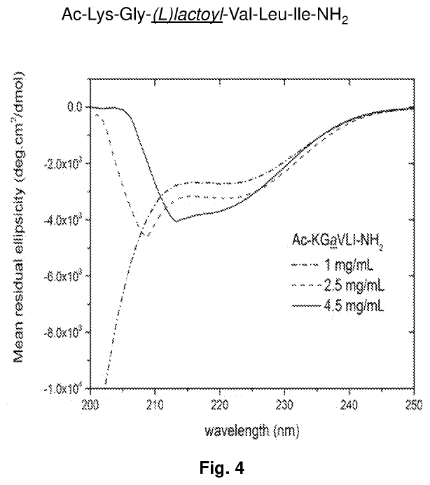

Interestingly, the inverted sequence with a free N-terminus KGAVLI-NH₂ also undergoes the same conformational transitions (FIG. 3a). In the original Gen-1 motif, acetylation is integral to self-assembly. Without it, peptides do not self-assemble, possibly due to the ionization of the free amine group at the N-terminus which leads to unfavourable charge interactions with the other peptides that discourage self-assembly. In eliminating the need for N-terminal acetylation, the inverted motif significantly widens the field of candidates accessible for biomedical applications. It would offer better solubility profiles and stimuli-enhanced gelation for peptide subclasses with neutral residues as the polar moiety. More importantly, it implicates that polar, bioactive moieties which are not amino acids can be substituted into the assembling motif to generate functionalized assemblies. Solid phase peptide synthesis occurs from C- to N-terminus. As the last moiety to be coupled, the functional groups on the N-terminal moiety may not require as extensive protection, making the overall synthesis easier. The self-assembling motif can thus be harnessed for the display of bioactive epitopes. In contrast, the chemistry of the moiety-of-interest may not lend itself to facile coupling onto the resin, which is needed for preparing Gen-1 peptides. The hypothesis is supported by observations that Ac-GAVLI-NH₂, representatives from the serine subclass (Ac-SGAVIA-NH₂ and SGAVLI-NH₂) and histidine subclass (Ac-HGAVLI-NH₂ and Ac-HGAVIA-NH₂) all formed thixotrophic gels in dimethylsulfoxide (FIG. 3b). In particular, the gelation behavior of Ac-GAVLI-NH₂ suggests that the polar moiety need not be an amino acid and can be fulfilled by N-acetylation or other polar functional groups. By extension, depsipeptide analogs of the inverted motif also self-assemble into hydrogels in a stimuli-responsive fashion (FIG. 4a). These candidates were prepared by substituting the second (glycine) or third (L-alanine) amino acid with their alpha-hydroxy acid analogs (glycolic acid and L-lactic acid), giving rise to Ac-KgAVLI-NH₂ and Ac-KGaVLI-NH₂. In doing so, the N-terminus portions of the amide backbone are replaced with ester linkages. This results in reduced hydrogen bonding, as ester bonds are hydrogen acceptors but not donors. Despite so, circular dichroism spectra of these depsipeptide analogs show the same secondary structure transitions from random coil to α-helical and subsequently to β-type structures with increasing concentration. (FIG. 4b). By expanding the library of self-assembling sequences to encompass both non-acetylated peptides and depsipeptides, subclasses of motifs with better biodegradability can be defined, as N-terminal acetylation limits enzymatic degradation while ester bonds are more labile compared to amide bonds.

The inverted hexapeptide analogs self-assembled into nanofibrous hydrogels (FIG. 1c,d), similar to their Gen-1 counterparts. The gelation behavior is likewise enhanced by the addition of buffered salts and by increasing pH. Faster gelation kinetics at lower peptide concentrations were observed following mixing with phosphate-buffered saline (see Table 1 below).

TABLE 1

| | | Secondary | Minimum gelation |
|---|---|---|---|
| | Solubility | structure | concentration in |
| Peptide | in water | transitions | buffered saline |
| Ac-ILVAGK-NH₂ | ++ | Yes | 3 mg/mL |
| Ac-KGAVLI-NH₂ | +++ | Yes | 3 mg/mL |
| KGAVLI-NH₂ | +++ | Yes | 20 mg/mL |
| Ac-IVK-NH₂ | ++++ | Yes | >30 mg/mL |
| Ac-KVI-NH₂ | ++++ | Yes | >30 mg/mL |

The inverted sequence motif demonstrated better solubility in water and similar gelation behavior compared to the original motif. Both motifs demonstrate salt- and pH-enhanced gelation, having lower gelation concentrations in buffered saline. Unlike the original motif, N-terminal acetylation is not a pre-requisite for self-assembly as KGAVLI-NH₂ with its free amine terminus also undergoes the same secondary structure transitions and forms hydrogels in buffered saline. In general, hexamer peptides demonstrate better gelation with lower minimum gelation concentrations.

The storage moduli of hydrogels prepared in buffered saline are also comparable to Ac-ILVAGK-NH₂ at 10 kPa magnitude (FIG. 1e). Hexameric peptides are by far superior gelators, as the tripeptides remained solutions in all the conditions evaluated. It is postulated that self-assembly and by extension gelation, is a delicate balance between solubility and aggregation. The observations thus underscore the importance of the hydrophobic contribution towards self-assembly.

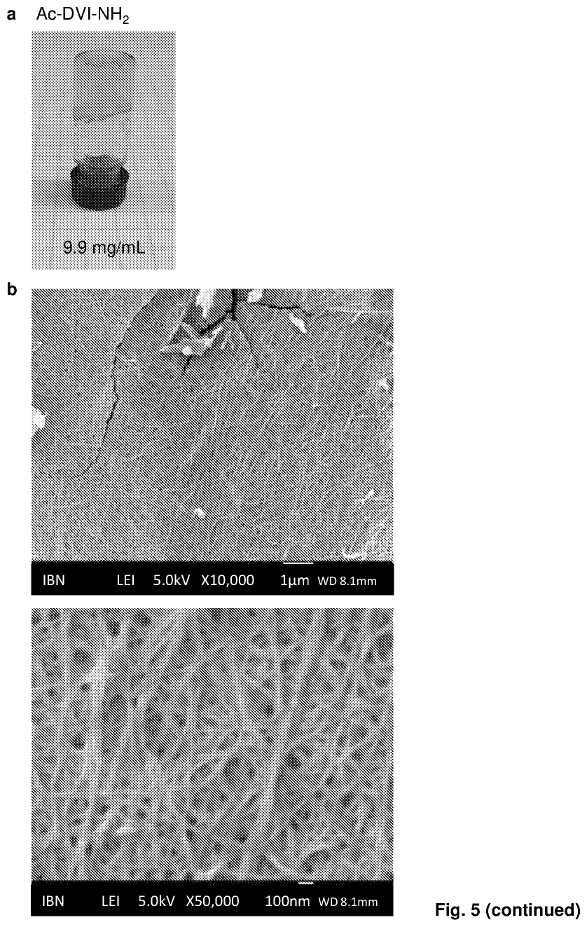

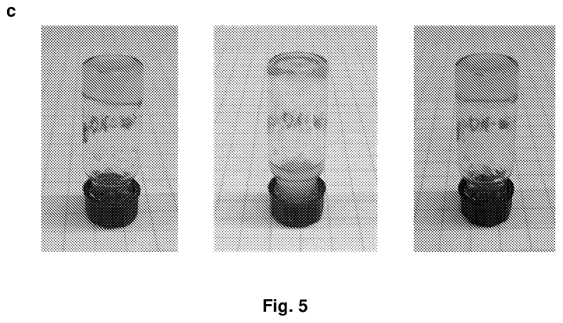

Expanding the observations beyond the lysine subclass, representative candidates with acidic and neutral polar moieties were explored. In particular, unlike its Gen-1 counterparts, Ac-DVI-NH₂ bears C-terminus amidation to avoid zwitterion formation (Table 2 and FIG. 5). This inhibited solvation in physiologically buffered solutions, allowing the peptide to self-assemble into nanofibrous scaffolds (Table 2 and FIG. 5b).

TABLE 2

| | | | TGA Degradation |
|---|---|---|---|
| Peptide | [Peptide] _{min, water} | [Peptide] _{min, PBS} | Temperature (° C.) |
| Ac-IVD | 40.8 mg/mL | 19.2 mg/mL | 323.6 |
| Ac-IVD-NH₂ | 44.9 mg/mL | 33.9 mg/mL | 328.5 |
| Ac-DVI-NH₂ | 11.6 mg/mL | 9.9 mg/mL | 321.5 |

The resulting hydrogel is thixotrophic (FIG. 5c). This observation is contrary to computational simulations previously developed by Smadbeck et al. In their study, a two-stage computation design framework which incorporates metrics for potential energy, fold specificity and approximate association affinity was applied to the design of ultrashort peptides. Ac-IVD was designated as the structural template for generating novel tripeptide hydrogel candidates. The inverted sequences were not amongst the shortlisted promising candidates. Further validations that included shuffled sequence variations suggested that Ac-EVI, the only inverted sequence experimentally evaluated, may not form hydrogels. While it is plausible that C-terminus amidation may have an unexpectedly larger contribution towards assembly, the marked differences with the present experimental observations nonetheless reflect the disconnection between in silico and in vitro models. This highlights the need for deeper understanding of the parameters involved in peptide self-assembly, in order to build more predictive computational algorithms.

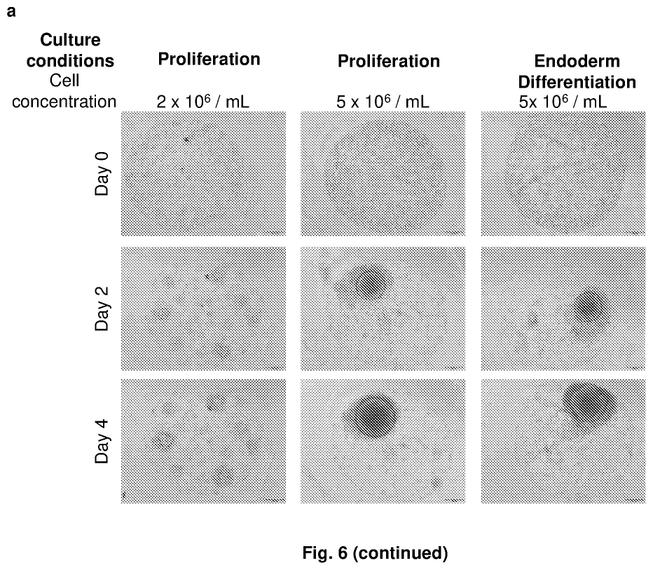

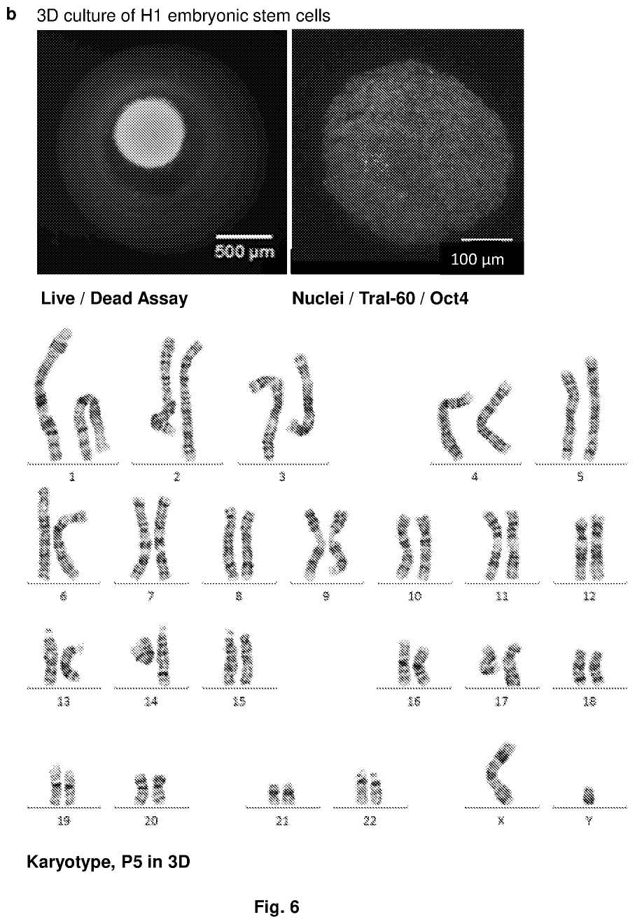

An unexpected advantage of inverting the printable Ac-ILVAGK-NH₂ Gen-1 sequence was improved biological properties for pluripotent stem cell culture. In a time-course study, cell behaviour was tracked in representative hydrogel droplets, and the same hydrogel droplets were imaged for six days. Using Ac-KGAVLI-NH₂ as the matrix for bioprinting hydrogel droplets encapsulating cells, higher retention of the expanding colonies was observed at day 7. Almost all of the hydrogel droplets were intact after 7 days of culture. More interestingly, at threshold cell encapsulation densities exceeding 5×10⁶ cells/m L, single cell suspensions of embryonic stem cells H1 and H9 were more likely to aggregate into a single colony when encapsulated in Ac-KGAVLI-NH₂ (FIG. 6a) whereas several colonies developed within Ac-ILVAGK-NH₂. The proportion of hydrogel droplets with just a single colony exceeded 95%. Lower cell seeding densities (2×10⁶ cells/mL) resulted in the formation of several colonies within a single droplet. The same observations were made when differentiation media was applied. When cultured in defined mTESR media, the cells retained their pluripotency, as evident from the staining of Oct4 and Tral-60 biomarkers (FIG. 6b). The peptide was non-mutagenic, as evident from the lack of chromosomal aberrations after 5 passages of 3D culture (FIG. 6b). The enhanced stability, and more importantly, consistent and reproducible conditions for single colony development makes Ac-KGAVLI-NH₂ hydrogels ideal matrices for stem cell differentiation into organoids. Stem cell can thus be encapsulated within Ac-KGAVLI-NH₂ hydrogel droplets for long term culture. Coupled with its printability, this peptide can be exploited for automation of stem cell culture and “one-pot” organoid derivation for high-throughput screening of therapeutics. Lower cell encapsulation densities tended to give rise to several colonies within a single drop and would thus be more useful for stem cell expansion.

The example self-assembling sequences are shown in Table 3 below.

TABLE 3

| | Basic | Neutral | Acidic |
|---|---|---|---|
| | Amino | Amino | Amino |
| | Acid | Acid | Acid |
| | Constituents | Constituents | Constituents |
| N-terminal | Ac-KVI-NH₂ | Ac-GAVLI-NH₂ | Ac-DVI-NH₂ |
| acetylated | Ac-KGAVLI-NH₂ | Ac-SGAVLI-NH₂ | Ac-DGAVLI-NH₂ |
| | Ac-KGAVIL-NH₂ | Ac-SGAVIL-NH₂ | Ac-DGAVIL-NH₂ |
| | Ac-KGAVIA-NH₂ | Ac-SGAVIA-NH₂ | Ac-EVI-NH₂ |
| | Ac-RVI-NH₂ | Ac-TGAVLI-NH₂ | Ac-EGAVLI-NH₂ |
| | Ac-RGAVLI-NH₂ | Ac-TGAVIL-NH₂ | Ac-EGAVIL-NH₂ |
| | Ac-RGAVIL-NH₂ | Ac-TGAVIA-NH₂ | Ac-EGAVIA-NH₂ |
| | Ac-RGAVIA-NH₂ | | |
| | Ac-HVI-NH₂ | | |
| | Ac-HGAVLI-NH₂ | | |
| | Ac-HGAVIL-NH₂ | | |
| | Ac-HGAVIA-NH₂ | | |
| | Ac-OrnVI-NH₂ | | |
| | Ac-OrnGAVLI-NH₂ | | |
| | Ac-OrnGAVIL-NH₂ | | |
| | Ac-OrnGAVIA-NH₂ | | |
| | Ac-DapVI-NH₂ | | |
| | Ac-DapGAVLI-NH₂ | | |
| | Ac-DapGAVIL-NH₂ | | |
| | Ac-DapGAVIA-NH₂ | | |
| | Ac-DabVI-NH₂ | | |
| | Ac-DabGAVLI-NH₂ | | |
| | Ac-DabGAVIL-NH₂ | | |
| | Ac-DabGAVIA-NH₂ | | |
| Free N- | KGAVLI-NH₂ | SGAVLI-NH₂ | |
| terminus | KGAVIL-NH₂ | SGAVIA-NH₂ | |
| Depsipeptides | Ac-KgAVLI-NH₂ | Ac-SgAVLI-NH₂ | |
| | Ac-KGaVLI-NH₂ | Ac-SGaVLI-NH₂ | |
| | KgAVLI-NH₂ | SgAVLI-NH₂ | |
| | KGaVLI-NH₂ | SGaVLI-NH₂ | |
| | Ac-KgAVIL-NH₂ | Ac-SgAVIA-NH₂ | |
| | Ac-KGaVIL-NH₂ | Ac-SGaVIA-NH₂ | |
| | KgAVIL-NH₂ | SgAVIA-NH₂ | |
| | KGaVIL-NH₂ | SGaVIA-NH₂ | |

### EXAMPLES

### Amphiphilic Short Peptide Motif for Wound Healing

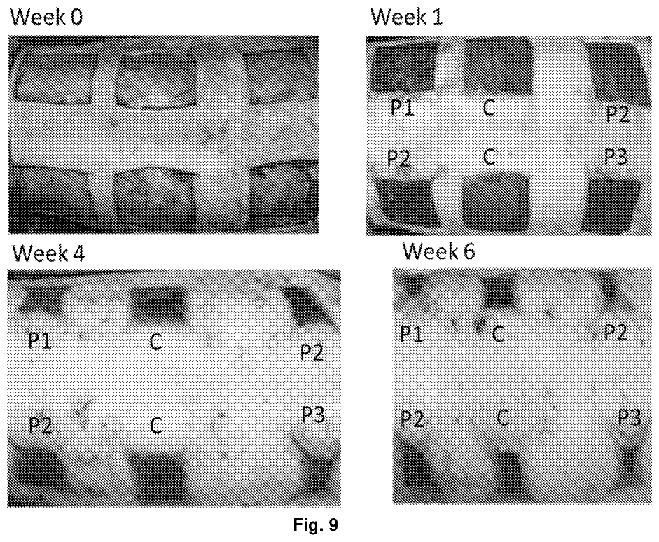

A porcine wound healing study was performed to evaluate the peptide hydrogels for wound healing applications. An 8 cm×8 cm excisional full thickness wound was created on the back of the animal, and three of the peptide hydrogels were applied using a polydimethylsiloxane mould. Wound dressing changes were carried out weekly for 6 weeks, with application of fresh hydrogel. The results point towards an effective reduction of wound size for all three peptides tested (i.e. P1: Ac-HGAVLI-NH₂; P2: SGAVLI-NH₂; P3: Ac-KGAVLI-NH₂). Representative images of the original wound sites and appearance after 1, 4 and 6 weeks are shown in FIG. 9.

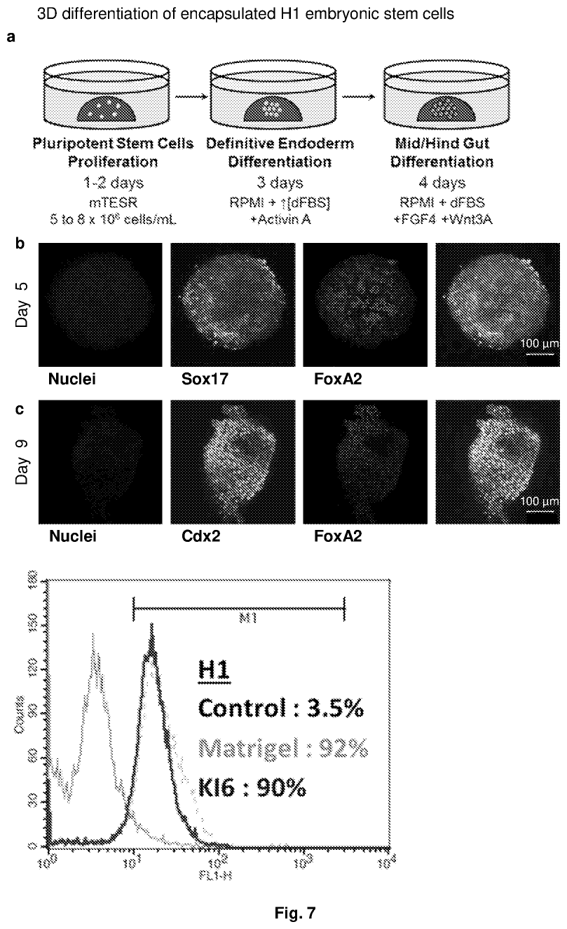

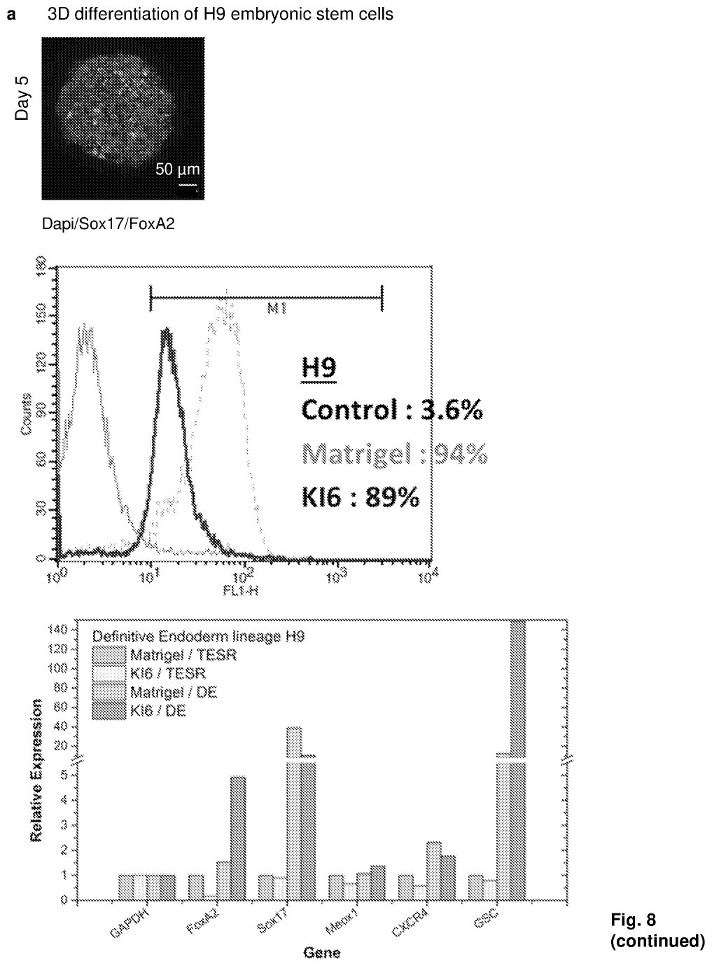

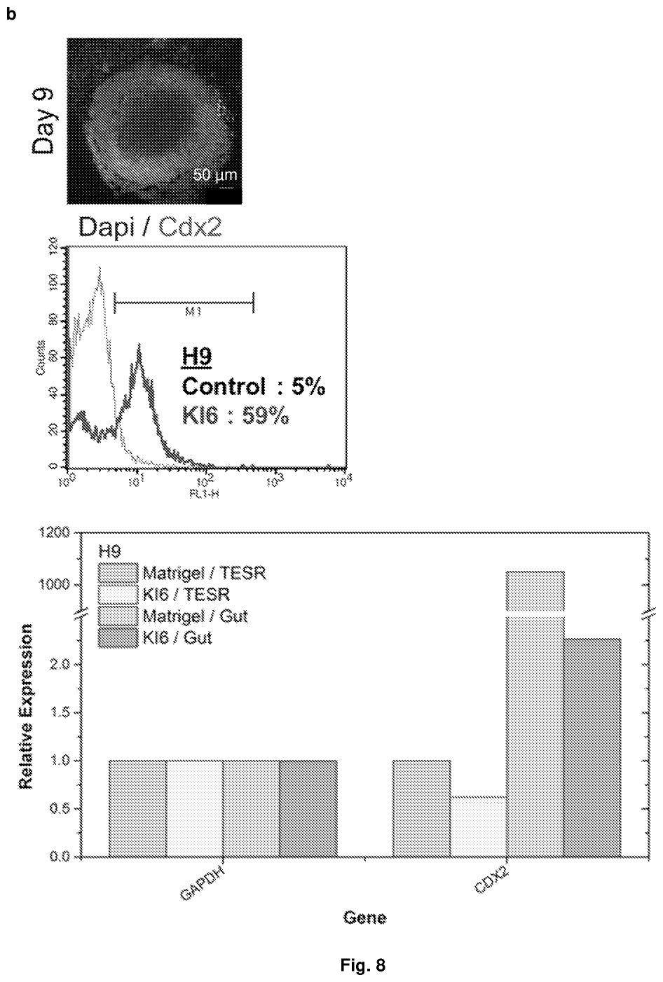

In recent years, stem cell-derived organoid cultures are increasingly being used as models to study tissue and disease development, and evaluate therapeutic candidates. In particular, the use of patient-derived organoids has revolutionized personalized medicine as they are superior predictive in vitro models. Intestinal organoids prepared from colon biopsies have successfully been used to identify patients who will respond to an experimental cystic fibrosis therapy. The development of such organoid models was boosted by advances in defined growth factor cocktails, which mimic the various stem cell niches. The biochemical cues stimulate the self-organization of cells into structures that partially recapitulate key tissue traits such as the spatial arrangement of heterogeneous cells, cellular interactions and some biological processes. A crucial step for organoid development is the encapsulation of stem cell-derived progenitors or adult stem cells into Matrigel (solubilized basement membrane preparation extracted from murine sarcoma). This suggests that cell-substrate interactions in a 3D microenvironment are integral for cell migration during organoid differentiation. Due to its nanofibrous macromolecular architecture, ultrashort peptide hydrogels would be an ideal substitute for Matrigel. When paired with defined media, peptide hydrogels constitute a completely defined culture environment, free of xenogenic components. Although such a synthetic matrix would be devoid of natural ligands, bioactive motifs can be easily incorporated using various conjugation strategies. This will confer better control over the biochemical makeup of the microenvironment, and predispose differentiation into preferred lineages. As a proof-of-concept, the protocol described by Spence et al. was adapted for the direct 3D differentiation of embryonic stem cells into hindgut spheroids (FIG. 7a). The colonies were first differentiated into definitive endoderm by applying Activin A with increasing concentrations of defined serum. Definitive endoderm induction was verified by confocal imaging of Sox17 and FoxA2 biomarkers (FIG. 7b, FIG. 8a). 90% of the cells express Sox17, comparable to 92% of cells differentiated under 2D (Matrigel culture) conditions. Further differentiation into mid- and hindgut was achieved through application of high concentrations of FGF4 and Wnt3A, as seen from the expression of Cdx2 biomarker (FIG. 7c, FIG. 8b).

The motif is more important than sequence in dictating the self-assembly of ultrashort peptides. While the physicochemical properties are largely unchanged, sequence inversion of ultrashort self-assembling peptides can have significant effect on its biological properties. Most notably, when used as synthetic 3D stem cell culture substrates, it was observed that Ac-KGAVLI-NH₂ prompted enhanced cell migration and better consistency in generating single colonies for reproducible organoid derivation, compared to its Gen1 analog. Its stimuli-responsive gelation properties can be harnessed for bioprinting, to reproducibly encapsulate cells for organoid differentiation. This scalable and customizable manufacturing technique can be automated for large-scale culture or for generating defined multi-domain tissue constructs for screening therapeutics, studying disease development and elucidating cellular interactions.

It will be appreciated by a person skilled in the art that other variations and/or modifications may be made to the embodiments disclosed herein without departing from the spirit or scope of the disclosure as broadly described. For example, in the description herein, features of different exemplary embodiments may be mixed, combined, interchanged, incorporated, adopted, modified, included etc. or the like across different exemplary embodiments. The present embodiments are, therefore, to be considered in all respects to be illustrative and not restrictive.

### REFERENCE

1. Mendes, A. C.; Baran, E. T.; Reis, R. L.; Azevedo, H. S., Self-assembly in nature: using the principles of nature to create complex nanobiomaterials. Wiley interdisciplinary reviews. Nanomedicine and nanobiotechnology 2013, 5 (6), 582-612.
2. Loo, Y.; Zhang, S.; Hauser, C. A., From short peptides to nanofibers to macromolecular assemblies in biomedicine. Biotechnol Adv 2012, 30 (3), 593-603.
3. (a) Fallas, J. A.; O'Leary, L. E.; Hartgerink, J. D., Synthetic collagen mimics: self-assembly of homotrimers, heterotrimers and higher order structures. Chem Soc Rev 2010, 39 (9), 3510-27; (b) Gauba, V.; Hartgerink, J. D., Self-assembled heterotrimeric collagen triple helices directed through electrostatic interactions. J Am Chem Soc 2007, 129 (9), 2683-90.
4. (a) Bromley, E. H.; Sessions, R. B.; Thomson, A. R.; Woolfson, D. N., Designed alpha-helical tectons for constructing multicomponent synthetic biological systems. J Am Chem Soc 2009, 131 (3), 928-30; (b) Papapostolou, D.; Smith, A. M.; Atkins, E. D.; Oliver, S. J.; Ryadnov, M. G.; Serpell, L. C.; Woolfson, D. N., Engineering nanoscale order into a designed protein fiber. Proc. Natl. Acad. Sci. USA 2007, 104 (26), 10853-8.
5. (a) Frederix, P.; Patmanidis, I.; Marrink, S. J., Molecular simulations of self-assembling bio-inspired supramolecular systems and their connection to experiments. Chemical Society reviews 2018, 47 (10), 3470-3489; (b) Makam, P.; Gazit, E., Minimalistic peptide supramolecular co-assembly: expanding the conformational space for nanotechnology. Chemical Society reviews 2018, 47 (10), 3406-3420.
6. (a) Zhang, S., Emerging biological materials through molecular self-assembly. Biotechnol Adv 2002, 20 (5-6), 321-39; (b) Zhang, S.; Holmes, T.; Lockshin, C.; Rich, A., Spontaneous assembly of a self-complementary oligopeptide to form a stable macroscopic membrane. Proc. Natl. Acad. Sci. USA 1993, 90 (8), 3334-8.
7. Loo, Y.; Lakshmanan, A.; Ni, M.; Toh, L. L.; Wang, S.; Hauser, C. A., Peptide Bioink: Self-Assembling Nanofibrous Scaffolds for Three-Dimensional Organotypic Cultures. Nano letters 2015, 15 (10), 6919-25.
8. Hauser, C. A.; Deng, R.; Mishra, A.; Loo, Y.; Khoe, U.; Zhuang, F.; Cheong, D. W.; Accardo, A.; Sullivan, M. B.; Riekel, C.; Ying, J. Y.; Hauser, U. A., Natural tri- to hexapeptides self-assemble in water to amyloid beta-type fiber aggregates by unexpected alpha-helical intermediate structures. Proc Natl Acad Sci USA 2011, 108 (4), 1361-6.
9. Smadbeck, J.; Chan, K. H.; Khoury, G. A.; Xue, B.; Robinson, R. C.; Hauser, C. A.; Floudas, C. A., De novo design and experimental characterization of ultrashort self-associating peptides. Accounts of chemical research 2014, 10 (7), e1003718.
10. Clevers, H., Modeling Development and Disease with Organoids. Cell 2016, 165 (7), 1586-1597.
11. (a) Saini, A., Cystic Fibrosis Patients Benefit from Mini Guts. Cell Stem Cell 19 (4), 425-427; (b) Dekkers, J. F.; Wiegerinck, C. L.; de Jonge, H. R.; Bronsveld, I.; Janssens, H. M.; de Winter-de Groot, K. M.; Brandsma, A. M.; de Jong, N. W.; Bijvelds, M. J.; Scholte, B. J.; Nieuwenhuis, E. E.; van den Brink, S.; Clevers, H.; van der Ent, C. K.; Middendorp, S.; Beekman, J. M., A functional CFTR assay using primary cystic fibrosis intestinal organoids. Nature medicine 2013, 19 (7), 939-45.
12. Yin, X.; Mead, B. E.; Safaee, H.; Langer, R.; Karp, J. M.; Levy, O., Engineering Stem Cell Organoids. Cell Stem Cell 2016, 18 (1), 25-38.
13. Spence, J. R.; Mayhew, C. N.; Rankin, S. A.; Kuhar, M. F.; Vallance, J. E.; Tolle, K.; Hoskins, E. E.; Kalinichenko, V. V.; Wells, S. I.; Zorn, A. M.; Shroyer, N. F.; Wells, J. M., Directed differentiation of human pluripotent stem cells into intestinal tissue in vitro. Nature 2011, 470 (7332), 105-9.

### APPLICATIONS

Peptide self-assembly is driven by secondary structure and intermolecular interactions, which are in turn dictated by peptide sequence. In view of the strict rules governing the design of helical self-assembling motifs, it is surprising that the self-assembly of ultrashort peptides into helical fibers is found to be unaffected by sequence inversion and the consequent reversal in peptide backbone direction.

During self-assembly, trimeric and hexameric inverted sequences are observed to undergo the same secondary structure transitions as their parent sequences, forming rigid, nanofibrous hydrogels in physiologically buffered saline. The results suggest that motif is more important than sequence in dictating the self-assembly of ultrashort peptides.

While the physicochemical properties are largely unchanged, sequence inversion of ultrashort self-assembling peptides can have significant effect on its biological properties. Most notably, when used as synthetic 3D stem cell culture substrates, it was observed that Ac-KGAVLI-NH₂ prompted enhanced cell migration and consistency in generating single colonies for organoid derivation, compared to Ac-ILVAGK-NH₂. In view of its reproducibility and printability, this new subset of self-organizing nanobiomaterials is well-positioned to facilitate the bioengineering of scalable and customizable in vitro tissue models.

1. An amphiphilic peptide having the formula (I):
XYZ (I)
wherein
X is a polar moiety at the N-terminus;
Y and Z each independently has between 1 to 4 residues of aliphatic amino acids or analogs or derivatives thereof, and
wherein the average degree of hydrophobicity of the residues in block Z is more than the average degree of hydrophobicity of the residues in block Y.

2. The amphiphilic peptide of claim 1, wherein the polar moiety is selected from the group consisting of a polar functional group, a polar amino acid, and a small polar biomolecule.

3. The amphiphilic peptide of claim 1, wherein the polar moiety is selected from the group consisting of a polar functional group, a polar amino acid, and a small polar biomolecule, wherein
the polar functional group is selected from the group consisting of amine, acetyl, hydroxyl, thiol, maleimide, and acid; or
the polar amino acid is selected from the group consisting of lysine, histidine, glycine, serine and aspartic acid; or
the small polar biomolecule is selected from the group consisting of biotin, alcohol, and saccharide.

4. The amphiphilic peptide of claim 1, wherein the amphiphilic peptide comprises a depsipeptide analog.

5. The amphiphilic peptide of claim 1, wherein the amphiphilic peptide comprises a depsipeptide analog, wherein depsipeptide analog comprises an α-hydroxy acid analog.

6. The amphiphilic peptide of claim 1, wherein the N-terminus is acetylated and/or C-terminus is amidated.

7. The amphiphilic peptide of claim 1, wherein the aliphatic amino acids comprise D-amino acids.

8. The amphiphilic peptide of claim 1, wherein the chirality of each of the residues in Z is the same.

9. The amphiphilic peptide of claim 1, wherein Z comprises a residue of an aliphatic amino acid with hydrophobic side chain, or analogs or derivatives thereof.

10. The amphiphilic peptide of claim 1, wherein the amphiphilic peptide is no more than 7 residues in length.

11. The amphiphilic peptide of claim 1, wherein the amphiphilic peptide is capable of self-assembling into a hydrogel.

12. The amphiphilic peptide of claim 1, wherein the amphiphilic peptide is selected from the group consisting of:
KVI, KGAVLI, KGAVIL, KGAVIA, RVI, RGAVLI, RGAVIL, RGAVIA, HVI, HGAVLI, HGAVIL, HGAVIA, OrnVI, OrnGAVLI, OrnGAVIL, OrnGAVIA, DapVI, DapGAVLI, DapGAVIL, DapGAVIA, Dab VI, DabGAVLI, DabGAVIL, DabGAVIA, KgAVLI, KGaVLI, KgAVIL, KGaVIL;
KGAVLI-NH₂, KGAVIL-NH₂, KgAVLI-NH₂, KGaVLI-NH₂, KgAVIL-NH₂, and KGaVIL-NH₂;
Ac-KVI-NH₂, Ac-KGAVLI-NH₂, Ac-KGAVIL-NH₂, Ac-KGAVIA-NH₂, Ac-RVI-NH₂, Ac-RGAVLI-NH₂, Ac-RGAVIL-NH₂, Ac-RGAVIA-NH₂, Ac-HVI-NH₂, Ac-HGAVLI-NH₂, Ac-HGAVIL-NH₂, Ac-HGAVIA-NH₂, Ac-OrnVI-NH₂, Ac-OrnGAVLI-NH₂, Ac-OrnGAVIL-NH₂, Ac-OrnGAVIA-NH₂, Ac-DapVI-NH₂, Ac-DapGAVLI-NH₂, Ac-DapGAVIL-NH₂, Ac-DapGAVIA-NH₂, Ac-DabVI-NH₂, Ac-DabGAVLI-NH₂, Ac-DabGAVIL-NH₂, Ac-DabGAVIA-NH₂, Ac-KgAVLI-NH₂, Ac-KGaVLI-NH₂, Ac-KgAVIL-NH₂, Ac-KGaVIL-NH₂;
GAVLI, SGAVLI, SGAVIL, SGAVIA, TGAVLI, TGAVIL, TGAVIA, SGAVLI, SGAVIA, SgAVLI, SGaVLI, SgAVIA, and SGaVIA;
SGAVLI-NH₂, SGAVIA-NH₂, SgAVLI-NH₂, SGaVLI-NH₂, SgAVIA-NH₂, and SGaVIA-NH₂;
Ac-GAVLI-NH₂, Ac-SGAVLI-NH₂, Ac-SGAVIL-NH₂, Ac-SGAVIA-NH₂, Ac-TGAVLI-NH₂, Ac-TGAVIL-NH₂, Ac-TGAVIA-NH₂, Ac-SgAVLI-NH₂, Ac-SGaVLI-NH₂, Ac-SgAVIA-NH₂, Ac-SGaVIA-NH₂;
DVI, DGAVLI, DGAVIL, EVI, EGAVLI, EGAVIL, and EGAVIA; and
Ac-DVI-NH₂, Ac-DGAVLI-NH₂, Ac-DGAVIL-NH₂, Ac-EVI-NH₂, Ac-EGAVLI-NH₂, Ac-EGAVIL-NH₂, and Ac-EGAVIA-NH₂,
wherein Orn=ornithine, Dap=2,3-diaminopropionic acid, Dab=2,4-diaminobutyric acid, g=glycolic acid and a=L-lactic acid.

13. The amphiphilic peptide of claim 1 comprised in a composition or a hydrogel.

14. The amphiphilic peptide of claim 1, wherein amphiphilic peptide has one or more properties selected from the group consisting of: stable, biocompatible, biodegradable, biomimetic, xenofree, injectable, thixotrophic, substantially non-mutagenic, substantially resistant to enzymatic degradation, responsive to stimulus, responsive to change in pH, responsive to change in salt concentration, responsive to change in temperature, compatible with bioprinting and has a storage modulus of at least 1 kPa.

15. (canceled)

16. A method of treating a subject in need of tissue regeneration, the method comprising administering the amphiphilic peptide of claim 1 or a composition or a hydrogel comprising the amphiphilic peptide into the subject in need thereof.

17. (canceled)

18. A method of cell, tissue or organoid culture, the method comprising:
culturing the cell, the tissue or the organoid in contact with the amphiphilic peptide of claim 1, or a composition or a hydrogel of the amphiphilic peptide.

19. The amphiphilic peptide of claim 1, wherein the amphiphilic peptide is comprised in a hydrogel and when single cells are seeded on or in the hydrogel, the hydrogel is more capable of promoting cell migration and/or generating single colony as compared to a hydrogel composed of peptides having sequences that are inverted from the sequences of said amphiphilic peptide.

20. The amphiphilic peptide of claim 1 when used in culturing stem cell, tissue or organoid.


SEQUENCE LISTING (106 sequences)

SEQ ID NO: 1 - protein, 3 aa, Artificial Sequence
  amphiphilic peptide
KVI

SEQ ID NO: 2 - protein, 6 aa, Artificial Sequence
  amphiphilic peptide
KGAVLI

SEQ ID NO: 3 - protein, 6 aa, Artificial Sequence
  amphiphilic peptide
KGAVIL

SEQ ID NO: 4 - protein, 6 aa, Artificial Sequence
  amphiphilic peptide
KGAVIA

SEQ ID NO: 5 - protein, 3 aa, Artificial Sequence
  amphiphilic peptide
RVI

SEQ ID NO: 6 - protein, 6 aa, Artificial Sequence
  amphiphilic peptide
RGAVLI

SEQ ID NO: 7 - protein, 6 aa, Artificial Sequence
  amphiphilic peptide
RGAVIL

SEQ ID NO: 8 - protein, 6 aa, Artificial Sequence
  amphiphilic peptide
RGAVIA

SEQ ID NO: 9 - protein, 3 aa, Artificial Sequence
  amphiphilic peptide
HVI

SEQ ID NO: 10 - protein, 6 aa, Artificial Sequence
  amphiphilic peptide
HGAVLI

SEQ ID NO: 11 - protein, 6 aa, Artificial Sequence
  amphiphilic peptide
HGAVIL

SEQ ID NO: 12 - protein, 6 aa, Artificial Sequence
  amphiphilic peptide
HGAVIA

SEQ ID NO: 13 - protein, 3 aa, Artificial Sequence
  amphiphilic peptide
  MOD_RES (1)..(1): Orn
XVI

SEQ ID NO: 14 - protein, 6 aa, Artificial Sequence
  amphiphilic peptide
  MOD_RES (1)..(1): Orn
XGAVLI

SEQ ID NO: 15 - protein, 6 aa, Artificial Sequence
  amphiphilic peptide
  MOD_RES (1)..(1): Orn
XGAVIL

SEQ ID NO: 16 - protein, 6 aa, Artificial Sequence
  amphiphilic peptide
  MOD_RES (1)..(1): Orn
XGAVIA

SEQ ID NO: 17 - protein, 3 aa, Artificial Sequence
  amphiphilic peptide
  MOD_RES (1)..(1): Dpr
XVI

SEQ ID NO: 18 - protein, 6 aa, Artificial Sequence
  amphiphilic peptide
  MOD_RES (1)..(1): Dpr
XGAVLI

SEQ ID NO: 19 - protein, 6 aa, Artificial Sequence
  amphiphilic peptide
  MOD_RES (1)..(1): Dpr
XGAVIL

SEQ ID NO: 20 - protein, 6 aa, Artificial Sequence
  amphiphilic peptide
  MOD_RES (1)..(1): Dpr
XGAVIA

SEQ ID NO: 21 - protein, 3 aa, Artificial Sequence
  amphiphilic peptide
  MOD_RES (1)..(1): Dbu
XVI

SEQ ID NO: 22 - protein, 6 aa, Artificial Sequence
  amphiphilic peptide
  MOD_RES (1)..(1): Dbu
XGAVLI

SEQ ID NO: 23 - protein, 6 aa, Artificial Sequence
  amphiphilic peptide
  MOD_RES (1)..(1): Dbu
XGAVIL

SEQ ID NO: 24 - protein, 6 aa, Artificial Sequence
  amphiphilic peptide
  MOD_RES (1)..(1): Dbu
XGAVIA

SEQ ID NO: 25 - protein, 6 aa, Artificial Sequence
  amphiphilic peptide
  Misc_feature (2)..(2): glycolic acid
KGAVLI

SEQ ID NO: 26 - protein, 6 aa, Artificial Sequence
  amphiphilic peptide
  Misc_feature (3)..(3): L-lactic acid
KGAVIL

SEQ ID NO: 27 - protein, 6 aa, Artificial Sequence
  amphiphilic peptide
  Misc_feature (2)..(2): glycolic acid
KGAVLI

SEQ ID NO: 28 - protein, 6 aa, Artificial Sequence
  amphiphilic peptide
  Misc_feature (3)..(3): L-lactic acid
KGAVLI

SEQ ID NO: 29 - protein, 6 aa, Artificial Sequence
  amphiphilic peptide
  MOD_RES (6)..(6): AMIDATION
KGAVLI

SEQ ID NO: 30 - protein, 6 aa, Artificial Sequence
  amphiphilic peptide
  MOD_RES (6)..(6): AMIDATION
KGAVLI

SEQ ID NO: 31 - protein, 6 aa, Artificial Sequence
  amphiphilic peptide
  MISC_FEATURE (2)..(2): glycolic acid
  MOD_RES (6)..(6): AMIDATION
KGAVIL

SEQ ID NO: 32 - protein, 6 aa, Artificial Sequence
  amphiphilic peptide
  MISC_FEATURE (3)..(3): L-lactic acid
  MOD_RES (6)..(6): AMIDATION
KGAVIL

SEQ ID NO: 33 - protein, 6 aa, Artificial Sequence
  amphiphilic peptide
  MISC_FEATURE (2)..(2): glycolic acid
  MOD_RES (6)..(6): AMIDATION
KGAVIL

SEQ ID NO: 34 - protein, 6 aa, Artificial Sequence
  amphiphilic peptide
  MISC_FEATURE (3)..(3): L-lactic acid
  MOD_RES (6)..(6): AMIDATION
KGAVIL

SEQ ID NO: 35 - protein, 3 aa, Artificial Sequence
  amphiphilic peptide
  MOD_RES (1)..(1): ACETYLATION
  MOD_RES (3)..(3): AMIDATION
KVI

SEQ ID NO: 36 - protein, 6 aa, Artificial Sequence
  Synthetic sequence
  MOD_RES (1)..(1): ACETYLATION
  MOD_RES (6)..(6): AMIDATION
KGAVLI

SEQ ID NO: 37 - protein, 6 aa, Artificial Sequence
  amphiphilic peptide
  MOD_RES (1)..(1): ACETYLATION
  MOD_RES (6)..(6): AMIDATION
KGAVIL

SEQ ID NO: 38 - protein, 6 aa, Artificial Sequence
  amphiphilic peptide
  MOD_RES (1)..(1): ACETYLATION
  MOD_RES (6)..(6): AMIDATION
KGAVIA

SEQ ID NO: 39 - protein, 3 aa, Artificial Sequence
  amphiphilic peptide
  MOD_RES (1)..(1): AMIDATION
  MOD_RES (3)..(3): ACETYLATION
RVI

SEQ ID NO: 40 - protein, 6 aa, Artificial Sequence
  amphiphilic peptide
  MOD_RES (1)..(1): ACETYLATION
  MOD_RES (6)..(6): AMIDATION
RGAVLI

SEQ ID NO: 41 - protein, 6 aa, Artificial Sequence
  amphiphilic peptide
  MOD_RES (1)..(1): ACETYLATION
  MOD_RES (6)..(6): AMIDATION
RGAVIL

SEQ ID NO: 42 - protein, 6 aa, Artificial Sequence
  amphiphilic peptide
  MOD_RES (1)..(1): ACETYLATION
  MOD_RES (6)..(6): AMIDATION
RGAVIA

SEQ ID NO: 43 - protein, 3 aa, Artificial Sequence
  amphiphilic peptide
  MOD_RES (1)..(1): ACETYLATION
  MOD_RES (3)..(3): AMIDATION
HVI

SEQ ID NO: 44 - protein, 6 aa, Artificial Sequence
  amphiphilic peptide
  MOD_RES (1)..(1): ACETYLATION
  MOD_RES (6)..(6): AMIDATION
HGAVLI

SEQ ID NO: 45 - protein, 6 aa, Artificial Sequence
  amphiphilic peptide
  MOD_RES (1)..(1): ACETYLATION
  MOD_RES (6)..(6): AMIDATION
HGAVIL

SEQ ID NO: 46 - protein, 6 aa, Artificial Sequence
  amphiphilic peptide
  MOD_RES (1)..(1): ACETYLATION
  MOD_RES (6)..(6): AMIDATION
HGAVIA

SEQ ID NO: 47 - protein, 3 aa, Artificial Sequence
  amphiphilic peptide
  MOD_RES (1)..(1): Orn
  MOD_RES (1)..(1): ACETYLATION
  MOD_RES (3)..(3): AMIDATION
XVI

SEQ ID NO: 48 - protein, 6 aa, Artificial Sequence
  amphiphilic peptide
  MOD_RES (1)..(1): Orn
  MOD_RES (1)..(1): ACETYLATION
  MOD_RES (6)..(6): AMIDATION
XGAVLI

SEQ ID NO: 49 - protein, 6 aa, Artificial Sequence
  amphiphilic peptide
  MOD_RES (1)..(1): Orn
  MOD_RES (1)..(1): ACETYLATION
  MOD_RES (6)..(6): AMIDATION
XGAVIL

SEQ ID NO: 50 - protein, 6 aa, Artificial Sequence
  amphiphilic peptide
  MOD_RES (1)..(1): Orn
  MOD_RES (1)..(1): ACETYLATION
  MOD_RES (6)..(6): AMIDATION
XGAVIA

SEQ ID NO: 51 - protein, 3 aa, Artificial Sequence
  amphiphilic peptide
  MOD_RES (1)..(1): ACETYLATION
  MOD_RES (1)..(1): Dpr
  MOD_RES (3)..(3): AMIDATION
XVI

SEQ ID NO: 52 - protein, 6 aa, Artificial Sequence
  amphiphilic peptide
  MOD_RES (1)..(1): Dpr
  MOD_RES (1)..(1): ACETYLATION
  MOD_RES (6)..(6): AMIDATION
XGAVLI

SEQ ID NO: 53 - protein, 6 aa, Artificial Sequence
  amphiphilic peptide
  MOD_RES (1)..(1): Dpr
  MOD_RES (1)..(1): ACETYLATION
  MOD_RES (6)..(6): AMIDATION
XGAVIL

SEQ ID NO: 54 - protein, 6 aa, Artificial Sequence
  amphiphilic peptide
  MOD_RES (1)..(1): Dpr
  MOD_RES (1)..(1): ACETYLATION
  MOD_RES (6)..(6): AMIDATION
XGAVIA

SEQ ID NO: 55 - protein, 3 aa, Artificial Sequence
  amphiphilic peptide
  MOD_RES (1)..(1): Dbu
  MOD_RES (1)..(1): ACETYLATION
  MOD_RES (3)..(3): AMIDATION
XVI

SEQ ID NO: 56 - protein, 6 aa, Artificial Sequence
  amphiphilic peptide
  MOD_RES (1)..(1): Dbu
  MOD_RES (1)..(1): ACETYLATION
  MOD_RES (6)..(6): AMIDATION
XGAVLI

SEQ ID NO: 57 - protein, 6 aa, Artificial Sequence
  amphiphilic peptide
  MOD_RES (1)..(1): Dbu
  MOD_RES (1)..(1): ACETYLATION
  MOD_RES (6)..(6): AMIDATION
XGAVIL

SEQ ID NO: 58 - protein, 6 aa, Artificial Sequence
  amphiphilic peptide
  MOD_RES (1)..(1): Dbu
  MOD_RES (1)..(1): ACETYLATION
  MOD_RES (6)..(6): AMIDATION
XGAVIA

SEQ ID NO: 59 - protein, 6 aa, Artificial Sequence
  amphiphilic peptide
  MOD_RES (1)..(1): ACETYLATION
  Misc_feature (2)..(2): glycolic acid
  MOD_RES (6)..(6): AMIDATION
KGAVLI

SEQ ID NO: 60 - protein, 6 aa, Artificial Sequence
  amphiphilic peptide
  MOD_RES (1)..(1): ACETYLATION
  Misc_feature (3)..(3): L-lactic acid
  MOD_RES (6)..(6): AMIDATION
KGAVLI

SEQ ID NO: 61 - protein, 6 aa, Artificial Sequence
  amphiphilic peptide
  MOD_RES (1)..(1): ACETYLATION
  Misc_feature (2)..(2): glycolic acid
  MOD_RES (6)..(6): AMIDATION
KGAVIL

SEQ ID NO: 62 - protein, 6 aa, Artificial Sequence
  amphiphilic peptide
  MOD_RES (1)..(1): ACETYLATION
  Misc_feature (3)..(3): L-lactic acid
  MOD_RES (6)..(6): AMIDATION
KGAVIL

SEQ ID NO: 63 - protein, 5 aa, Artificial Sequence
  amphiphilic peptide
GAVLI

SEQ ID NO: 64 - protein, 6 aa, Artificial Sequence
  amphiphilic peptide
SGAVLI

SEQ ID NO: 65 - protein, 6 aa, Artificial Sequence
  amphiphilic peptide
SGAVIL

SEQ ID NO: 66 - protein, 6 aa, Artificial Sequence
  amphiphilic peptide
SGAVIA

SEQ ID NO: 67 - protein, 6 aa, Artificial Sequence
  amphiphilic peptide
TGAVLI

SEQ ID NO: 68 - protein, 6 aa, Artificial Sequence
  amphiphilic peptide
TGAVIL

SEQ ID NO: 69 - protein, 6 aa, Artificial Sequence
  amphiphilic peptide
TGAVIA

SEQ ID NO: 70 - protein, 6 aa, Artificial Sequence
  amphiphilic peptide
SGAVLI

SEQ ID NO: 71 - protein, 6 aa, Artificial Sequence
  amphiphilic peptide
SGAVIA

SEQ ID NO: 72 - protein, 6 aa, Artificial Sequence
  amphiphilic peptide
  Misc_feature (2)..(2): glycolic acid
SGAVLI

SEQ ID NO: 73 - protein, 6 aa, Artificial Sequence
  amphiphilic peptide
  Misc_feature (3)..(3): L-lactic acid
SGAVLI

SEQ ID NO: 74 - protein, 6 aa, Artificial Sequence
  amphiphilic peptide
  Misc_feature (2)..(2): glycolic acid
SGAVIA

SEQ ID NO: 75 - protein, 6 aa, Artificial Sequence
  amphiphilic peptide
  Misc_feature (3)..(3): L-lactic acid
SGAVIA

SEQ ID NO: 76 - protein, 6 aa, Artificial Sequence
  amphiphilic peptide
  MOD_RES (6)..(6): AMIDATION
SGAVLI

SEQ ID NO: 77 - protein, 6 aa, Artificial Sequence
  amphiphilic peptide
  MOD_RES (6)..(6): AMIDATION
SGAVIA

SEQ ID NO: 78 - protein, 6 aa, Artificial Sequence
  amphiphilic peptide
  Misc_feature (2)..(2): glycolic acid
  MOD_RES (6)..(6): AMIDATION
SGAVLI

SEQ ID NO: 79 - protein, 6 aa, Artificial Sequence
  amphiphilic peptide
  Misc_feature (3)..(3): L-lactic acid
  MOD_RES (6)..(6): AMIDATION
SGAVLI

SEQ ID NO: 80 - protein, 6 aa, Artificial Sequence
  amphiphilic peptide
  Misc_feature (2)..(2): glycolic acid
  MOD_RES (6)..(6): AMIDATION
SGAVIA

SEQ ID NO: 81 - protein, 6 aa, Artificial Sequence
  amphiphilic peptide
  Misc_feature (3)..(3): L-lactic acid
  MOD_RES (6)..(6): AMIDATION
SGAVIA

SEQ ID NO: 82 - protein, 5 aa, Artificial Sequence
  amphiphilic peptide
  MOD_RES (1)..(1): ACETYLATION
  MOD_RES (5)..(5): AMIDATION
GAVLI

SEQ ID NO: 83 - protein, 6 aa, Artificial Sequence
  amphiphilic peptide
  MOD_RES (1)..(1): ACETYLATION
  MOD_RES (6)..(6): AMIDATION
SGAVLI

SEQ ID NO: 84 - protein, 6 aa, Artificial Sequence
  amphiphilic peptide
  MOD_RES (1)..(1): ACETYLATION
  MOD_RES (6)..(6): AMIDATION
SGAVIL

SEQ ID NO: 85 - protein, 6 aa, Artificial Sequence
  amphiphilic peptide
  MOD_RES (1)..(1): ACETYLATION
  MOD_RES (6)..(6): AMIDATION
SGAVIA

SEQ ID NO: 86 - protein, 6 aa, Artificial Sequence
  amphiphilic peptide
  MOD_RES (1)..(1): ACETYLATION
  MOD_RES (6)..(6): AMIDATION
TGAVLI

SEQ ID NO: 87 - protein, 6 aa, Artificial Sequence
  amphiphilic peptide
  MOD_RES (1)..(1): ACETYLATION
  MOD_RES (6)..(6): AMIDATION
TGAVIL

SEQ ID NO: 88 - protein, 6 aa, Artificial Sequence
  amphiphilic peptide
  MOD_RES (1)..(1): ACETYLATION
  MOD_RES (6)..(6): AMIDATION
TGAVIA

SEQ ID NO: 89 - protein, 6 aa, Artificial Sequence
  amphiphilic peptide
  MOD_RES (1)..(1): ACETYLATION
  Misc_feature (2)..(2): glycolic acid
  MOD_RES (6)..(6): AMIDATION
SGAVLI

SEQ ID NO: 90 - protein, 6 aa, Artificial Sequence
  amphiphilic peptide
  MOD_RES (1)..(1): ACETYLATION
  Misc_feature (3)..(3): L-lactic acid
  MOD_RES (6)..(6): AMIDATION
SGAVLI

SEQ ID NO: 91 - protein, 6 aa, Artificial Sequence
  amphiphilic peptide
  MOD_RES (1)..(1): ACETYLATION
  Misc_feature (2)..(2): glycolic acid
  MOD_RES (6)..(6): AMIDATION
SGAVIA

SEQ ID NO: 92 - protein, 6 aa, Artificial Sequence
  amphiphilic peptide
  MOD_RES (1)..(1): ACETYLATION
  Misc_feature (3)..(3): L-lactic acid
  MOD_RES (6)..(6): AMIDATION
SGAVIA

SEQ ID NO: 93 - protein, 3 aa, Artificial Sequence
  amphiphilic peptide
DVI

SEQ ID NO: 94 - protein, 6 aa, Artificial Sequence
  amphiphilic peptide
DGAVLI

SEQ ID NO: 95 - protein, 6 aa, Artificial Sequence
  amphiphilic peptide
DGAVIL

SEQ ID NO: 96 - protein, 3 aa, Artificial Sequence
  amphiphilic peptide
EVI

SEQ ID NO: 97 - protein, 6 aa, Artificial Sequence
  amphiphilic peptide
EGAVLI

SEQ ID NO: 98 - protein, 6 aa, Artificial Sequence
  amphiphilic peptide
EGAVIL

SEQ ID NO: 99 - protein, 6 aa, Artificial Sequence
  amphiphilic peptide
EGAVIA

SEQ ID NO: 100 - protein, 3 aa, Artificial Sequence
  amphiphilic peptide
  MOD_RES (1)..(1): ACETYLATION
  MOD_RES (3)..(3): AMIDATION
DVI

SEQ ID NO: 101 - protein, 6 aa, Artificial Sequence
  amphiphilic peptide
  MOD_RES (1)..(1): ACETYLATION
  MOD_RES (6)..(6): AMIDATION
DGAVLI

SEQ ID NO: 102 - protein, 6 aa, Artificial Sequence
  amphiphilic peptide
  MOD_RES (1)..(1): ACETYLATION
  MOD_RES (6)..(6): AMIDATION
DGAVIL

SEQ ID NO: 103 - protein, 3 aa, Artificial Sequence
  amphiphilic peptide
  MOD_RES (1)..(1): ACETYLATION
  MOD_RES (3)..(3): AMIDATION
EVI

SEQ ID NO: 104 - protein, 6 aa, Artificial Sequence
  amphiphilic peptide
  MOD_RES (1)..(1): ACETYLATION
  MOD_RES (6)..(6): AMIDATION
EGAVLI

SEQ ID NO: 105 - protein, 6 aa, Artificial Sequence
  amphiphilic peptide
  MOD_RES (1)..(1): ACETYLATION
  MOD_RES (6)..(6): AMIDATION
EGAVIL

SEQ ID NO: 106 - protein, 6 aa, Artificial Sequence
  amphiphilic peptide
  MOD_RES (1)..(1): ACETYLATION
  MOD_RES (6)..(6): AMIDATION
EGAVIA